SEQUENCE LISTING (56 sequences)

SEQ ID NO: 1 - DNA, 63 bp, synthetic construct
  source 1..63: mol_type = other DNA
  misc_difference 34..36: note = n is a, c, g, or t
tcgtcggcagcgtcagatgtgtataagagacagnnntcgccttaagatgtgtataagagacag

SEQ ID NO: 2 - DNA, 63 bp, synthetic construct
  source 1..63: mol_type = other DNA
  misc_difference 34..36: note = n is a, c, g, or t
tcgtcggcagcgtcagatgtgtataagagacagnnnctagtacgagatgtgtataagagacag

SEQ ID NO: 3 - DNA, 63 bp, synthetic construct
  source 1..63: mol_type = other DNA
  misc_difference 34..36: note = n is a, c, g, or t
tcgtcggcagcgtcagatgtgtataagagacagnnnttctgcctagatgtgtataagagacag

SEQ ID NO: 4 - DNA, 63 bp, synthetic construct
  source 1..63: mol_type = other DNA
  misc_difference 34..36: note = n is a, c, g, or t
tcgtcggcagcgtcagatgtgtataagagacagnnngctcaggaagatgtgtataagagacag

SEQ ID NO: 5 - DNA, 63 bp, synthetic construct
  source 1..63: mol_type = other DNA
  misc_difference 34..36: note = n is a, c, g, or t
tcgtcggcagcgtcagatgtgtataagagacagnnnaggagtccagatgtgtataagagacag

SEQ ID NO: 6 - DNA, 63 bp, synthetic construct
  source 1..63: mol_type = other DNA
  misc_difference 34..36: note = n is a, c, g, or t
tcgtcggcagcgtcagatgtgtataagagacagnnncatgcctaagatgtgtataagagacag

SEQ ID NO: 7 - DNA, 63 bp, synthetic construct
  source 1..63: mol_type = other DNA
  misc_difference 34..36: note = n is a, c, g, or t
tcgtcggcagcgtcagatgtgtataagagacagnnngtagagagagatgtgtataagagacag

SEQ ID NO: 8 - DNA, 63 bp, synthetic construct
  source 1..63: mol_type = other DNA
  misc_difference 34..36: note = n is a, c, g, or t
tcgtcggcagcgtcagatgtgtataagagacagnnncagcctcgagatgtgtataagagacag

SEQ ID NO: 9 - DNA, 63 bp, synthetic construct
  source 1..63: mol_type = other DNA
  misc_difference 34..36: note = n is a, c, g, or t
tcgtcggcagcgtcagatgtgtataagagacagnnntgcctcttagatgtgtataagagacag

SEQ ID NO: 10 - DNA, 63 bp, synthetic construct
  source 1..63: mol_type = other DNA
  misc_difference 34..36: note = n is a, c, g, or t
tcgtcggcagcgtcagatgtgtataagagacagnnntcctctacagatgtgtataagagacag

SEQ ID NO: 11 - DNA, 63 bp, synthetic construct
  source 1..63: mol_type = other DNA
  misc_difference 34..36: note = n is a, c, g, or t
tcgtcggcagcgtcagatgtgtataagagacagnnntcatgagcagatgtgtataagagacag

SEQ ID NO: 12 - DNA, 63 bp, synthetic construct
  source 1..63: mol_type = other DNA
  misc_difference 34..36: note = n is a, c, g, or t
tcgtcggcagcgtcagatgtgtataagagacagnnncctgagatagatgtgtataagagacag

SEQ ID NO: 13 - DNA, 63 bp, synthetic construct
  source 1..63: mol_type = other DNA
  misc_difference 34..36: note = n is a, c, g, or t
tcgtcggcagcgtcagatgtgtataagagacagnnntagcgagtagatgtgtataagagacag

SEQ ID NO: 14 - DNA, 63 bp, synthetic construct
  source 1..63: mol_type = other DNA
  misc_difference 34..36: note = n is a, c, g, or t
tcgtcggcagcgtcagatgtgtataagagacagnnngtagctccagatgtgtataagagacag

SEQ ID NO: 15 - DNA, 63 bp, synthetic construct
  source 1..63: mol_type = other DNA
  misc_difference 34..36: note = n is a, c, g, or t
tcgtcggcagcgtcagatgtgtataagagacagnnntactacgcagatgtgtataagagacag

SEQ ID NO: 16 - DNA, 63 bp, synthetic construct
  source 1..63: mol_type = other DNA
  misc_difference 34..36: note = n is a, c, g, or t
tcgtcggcagcgtcagatgtgtataagagacagnnnaggctccgagatgtgtataagagacag

SEQ ID NO: 17 - DNA, 63 bp, synthetic construct
  source 1..63: mol_type = other DNA
  misc_difference 34..36: note = n is a, c, g, or t
tcgtcggcagcgtcagatgtgtataagagacagnnngcagcgtaagatgtgtataagagacag

SEQ ID NO: 18 - DNA, 63 bp, synthetic construct
  source 1..63: mol_type = other DNA
  misc_difference 34..36: note = n is a, c, g, or t
tcgtcggcagcgtcagatgtgtataagagacagnnnctgcgcatagatgtgtataagagacag

SEQ ID NO: 19 - DNA, 63 bp, synthetic construct
  source 1..63: mol_type = other DNA
  misc_difference 34..36: note = n is a, c, g, or t
tcgtcggcagcgtcagatgtgtataagagacagnnngagcgctaagatgtgtataagagacag

SEQ ID NO: 20 - DNA, 63 bp, synthetic construct
  source 1..63: mol_type = other DNA
  misc_difference 34..36: note = n is a, c, g, or t
tcgtcggcagcgtcagatgtgtataagagacagnnncgctcagtagatgtgtataagagacag

SEQ ID NO: 21 - DNA, 63 bp, synthetic construct
  source 1..63: mol_type = other DNA
  misc_difference 34..36: note = n is a, c, g, or t
tcgtcggcagcgtcagatgtgtataagagacagnnngtcttaggagatgtgtataagagacag

SEQ ID NO: 22 - DNA, 63 bp, synthetic construct
  source 1..63: mol_type = other DNA
  misc_difference 34..36: note = n is a, c, g, or t
tcgtcggcagcgtcagatgtgtataagagacagnnnactgatcgagatgtgtataagagacag

SEQ ID NO: 23 - DNA, 63 bp, synthetic construct
  source 1..63: mol_type = other DNA
  misc_difference 34..36: note = n is a, c, g, or t
tcgtcggcagcgtcagatgtgtataagagacagnnntagctgcaagatgtgtataagagacag

SEQ ID NO: 24 - DNA, 63 bp, synthetic construct
  source 1..63: mol_type = other DNA
  misc_difference 34..36: note = n is a, c, g, or t
tcgtcggcagcgtcagatgtgtataagagacagnnngacgtcgaagatgtgtataagagacag

SEQ ID NO: 25 - DNA, 63 bp, synthetic construct
  source 1..63: mol_type = other DNA
  misc_difference 34..36: note = n is a, c, g, or t
tcgtcggcagcgtcagatgtgtataagagacagnnnctctctatagatgtgtataagagacag

SEQ ID NO: 26 - DNA, 63 bp, synthetic construct
  source 1..63: mol_type = other DNA
  misc_difference 34..36: note = n is a, c, g, or t
tcgtcggcagcgtcagatgtgtataagagacagnnntatcctctagatgtgtataagagacag

SEQ ID NO: 27 - DNA, 63 bp, synthetic construct
  source 1..63: mol_type = other DNA
  misc_difference 34..36: note = n is a, c, g, or t
tcgtcggcagcgtcagatgtgtataagagacagnnngtaaggagagatgtgtataagagacag

SEQ ID NO: 28 - DNA, 63 bp, synthetic construct
  source 1..63: mol_type = other DNA
  misc_difference 34..36: note = n is a, c, g, or t
tcgtcggcagcgtcagatgtgtataagagacagnnnactgcataagatgtgtataagagacag

SEQ ID NO: 29 - DNA, 63 bp, synthetic construct
  source 1..63: mol_type = other DNA
  misc_difference 34..36: note = n is a, c, g, or t
tcgtcggcagcgtcagatgtgtataagagacagnnnaaggagtaagatgtgtataagagacag

SEQ ID NO: 30 - DNA, 63 bp, synthetic construct
  source 1..63: mol_type = other DNA
  misc_difference 34..36: note = n is a, c, g, or t
tcgtcggcagcgtcagatgtgtataagagacagnnnctaagcctagatgtgtataagagacag

SEQ ID NO: 31 - DNA, 63 bp, synthetic construct
  source 1..63: mol_type = other DNA
  misc_difference 34..36: note = n is a, c, g, or t
tcgtcggcagcgtcagatgtgtataagagacagnnncgtctaatagatgtgtataagagacag

SEQ ID NO: 32 - DNA, 63 bp, synthetic construct
  source 1..63: mol_type = other DNA
  misc_difference 34..36: note = n is a, c, g, or t
tcgtcggcagcgtcagatgtgtataagagacagnnntctctccgagatgtgtataagagacag

SEQ ID NO: 33 - DNA, 63 bp, synthetic construct
  source 1..63: mol_type = other DNA
  misc_difference 34..36: note = n is a, c, g, or t
tcgtcggcagcgtcagatgtgtataagagacagnnntcgactagagatgtgtataagagacag

SEQ ID NO: 34 - DNA, 63 bp, synthetic construct
  source 1..63: mol_type = other DNA
  misc_difference 34..36: note = n is a, c, g, or t
tcgtcggcagcgtcagatgtgtataagagacagnnnttctagctagatgtgtataagagacag

SEQ ID NO: 35 - DNA, 63 bp, synthetic construct
  source 1..63: mol_type = other DNA
  misc_difference 34..36: note = n is a, c, g, or t
tcgtcggcagcgtcagatgtgtataagagacagnnncctagagtagatgtgtataagagacag

SEQ ID NO: 36 - DNA, 63 bp, synthetic construct
  source 1..63: mol_type = other DNA
  misc_difference 34..36: note = n is a, c, g, or t
tcgtcggcagcgtcagatgtgtataagagacagnnngcgtaagaagatgtgtataagagacag

SEQ ID NO: 37 - DNA, 63 bp, synthetic construct
  source 1..63: mol_type = other DNA
  misc_difference 34..36: note = n is a, c, g, or t
tcgtcggcagcgtcagatgtgtataagagacagnnnctattaagagatgtgtataagagacag

SEQ ID NO: 38 - DNA, 63 bp, synthetic construct
  source 1..63: mol_type = other DNA
  misc_difference 34..36: note = n is a, c, g, or t
tcgtcggcagcgtcagatgtgtataagagacagnnnaaggctatagatgtgtataagagacag

SEQ ID NO: 39 - DNA, 63 bp, synthetic construct
  source 1..63: mol_type = other DNA
  misc_difference 34..36: note = n is a, c, g, or t
tcgtcggcagcgtcagatgtgtataagagacagnnngagccttaagatgtgtataagagacag

SEQ ID NO: 40 - DNA, 63 bp, synthetic construct
  source 1..63: mol_type = other DNA
  misc_difference 34..36: note = n is a, c, g, or t
tcgtcggcagcgtcagatgtgtataagagacagnnnttatgcgaagatgtgtataagagacag

SEQ ID NO: 41 - DNA, 63 bp, synthetic construct
  source 1..63: mol_type = other DNA
  misc_difference 34..36: note = n is a, c, g, or t
tcgtcggcagcgtcagatgtgtataagagacagnnntatagcctagatgtgtataagagacag

SEQ ID NO: 42 - DNA, 63 bp, synthetic construct
  source 1..63: mol_type = other DNA
  misc_difference 34..36: note = n is a, c, g, or t
tcgtcggcagcgtcagatgtgtataagagacagnnnatagaggcagatgtgtataagagacag

SEQ ID NO: 43 - DNA, 63 bp, synthetic construct
  source 1..63: mol_type = other DNA
  misc_difference 34..36: note = n is a, c, g, or t
tcgtcggcagcgtcagatgtgtataagagacagnnncctatcctagatgtgtataagagacag

SEQ ID NO: 44 - DNA, 63 bp, synthetic construct
  source 1..63: mol_type = other DNA
  misc_difference 34..36: note = n is a, c, g, or t
tcgtcggcagcgtcagatgtgtataagagacagnnnggctctgaagatgtgtataagagacag

SEQ ID NO: 45 - DNA, 63 bp, synthetic construct
  source 1..63: mol_type = other DNA
  misc_difference 34..36: note = n is a, c, g, or t
tcgtcggcagcgtcagatgtgtataagagacagnnnaggcgaagagatgtgtataagagacag

SEQ ID NO: 46 - DNA, 63 bp, synthetic construct
  source 1..63: mol_type = other DNA
  misc_difference 34..36: note = n is a, c, g, or t
tcgtcggcagcgtcagatgtgtataagagacagnnntaatcttaagatgtgtataagagacag

SEQ ID NO: 47 - DNA, 63 bp, synthetic construct
  source 1..63: mol_type = other DNA
  misc_difference 34..36: note = n is a, c, g, or t
tcgtcggcagcgtcagatgtgtataagagacagnnncaggacgtagatgtgtataagagacag

SEQ ID NO: 48 - DNA, 63 bp, synthetic construct
  source 1..63: mol_type = other DNA
  misc_difference 34..36: note = n is a, c, g, or t
tcgtcggcagcgtcagatgtgtataagagacagnnngtactgacagatgtgtataagagacag

SEQ ID NO: 49 - DNA, 41 bp, synthetic construct
  source 1..41: mol_type = other DNA
gtctcgtcgacgactgggctcgagatgtgtataagagacag

SEQ ID NO: 50 - DNA, 19 bp, synthetic construct
  source 1..19: mol_type = other DNA
ctgtctcttatacacatct

SEQ ID NO: 51 - DNA, 19 bp, synthetic construct
  source 1..19: mol_type = other DNA
agatgtgtataagagacag

SEQ ID NO: 52 - DNA, 52 bp, synthetic construct
  source 1..52: mol_type = other DNA
tcgtcggcagcgtcagatgtgtataagagacagagatgtgtataagagacag

SEQ ID NO: 53 - DNA, 33 bp, synthetic construct
  source 1..33: mol_type = other DNA
gtctcgtgggctcgagatgtgtataagagacag

SEQ ID NO: 54 - DNA, 29 bp, synthetic construct
  source 1..29: mol_type = other DNA
aatgatacggcgaccaccgagatctacac

SEQ ID NO: 55 - DNA, 34 bp, synthetic construct
  source 1..34: mol_type = other DNA
ctgtctcttatacacatctccgagcccacgagac

SEQ ID NO: 56 - DNA, 24 bp, synthetic construct
  source 1..24: mol_type = other DNA
atctcgtatgccgtcttctgcttg

(19) United States
(12) Patent Application Publication
(10) Pub. No.: US 2024/0043919 A1
(43) Pub. Date: Feb. 8, 2024

(54) METHOD FOR TRACEABLE MEDIUM-THROUGHPUT SINGLE-CELL COPY NUMBER SEQUENCING

(71) Applicant: Guangzhou SequMed Biology Technology Co., Ltd., Guangzhou (CN)

(72) Inventors: Xinghua Pan, Guangzhou (CN); Guanchuan Lin, Guangzhou (CN); Caiming Chen, Guangzhou (CN); Zhanying Dong, Guangzhou (CN)

(21) Appl. No.: 18/228,664

(22) Filed: Jul. 31, 2023

Related U.S. Application Data
(63) Continuation-in-part of application No. PCT/CN2022/073321, filed on Jan. 21, 2022.

(30) Foreign Application Priority Data
Feb. 1, 2021 (CN) 202110133128.5

(51) Int. Cl. C12Q 1/6869; C12Q 1/6844

(52) U.S. Cl. CPC C12Q 1/6869; C12Q 1/6844; C12Q 2600/156; C12Q 2600/16

(57) ABSTRACT

A method for construction of a medium-throughput single-cell copy number sequencing (MT-scCNV-seq) library and sequencing includes: delivering single cells each to a tube, and independently lysing each cell; labeling each cell with a cell-specific barcode while tagmenting the gDNA with an innovative Tn5 transposome; pooling the reactions of a plurality of cells simultaneously treated above, and constructing a batch of sequencing libraries for the cells collectively in a single tube with primers containing a batch index. The specific tagmentation of the gDNA of a given cell by the Tn5 transposome enables early pooling of multiple cells in a single tube for collective library construction, without pre-whole-genome-amplification of each cell. The output library is compatible with a conventional NGS platform and program. Finally the sequencing data is disaggregated, and traced to each cell according to the barcode and index; the CNV profile for each cell of the panel is accurately identified.

### CROSS-REFERENCE TO RELATED APPLICATIONS

The present application is a continuation-in-part application of PCT application No. PCT/CN2022/073321 filed on Jan. 21, 2022, which claims the benefit of Chinese Patent Application No. 202110133128.5 filed on Feb. 1, 2021. The contents of all of the aforementioned applications are incorporated by reference herein in their entirety.

### REFERENCE TO SEQUENCE LISTING

The Replacement Sequence Listing XML file submitted via the USPTO Patent Center, with a file name of “Replacement_Sequence_Listing_SCH-23116-USCIP.xml”, a creation date of Oct. 27, 2023, and a size of 70 KB, is part of the specification and is incorporated in its entirety by reference herein.

### TECHNICAL FIELD

The present application relates to the field of single-cell sequencing, and specifically to a method for single-cell copy number sequencing on medium-throughput scale (MT-scCNV-seq).

### BACKGROUND

With the vigorous development of human genome project and medicine, the next-generation sequencing (NGS) platform is increasingly mature and going to clinics. NGS includes genome sequencing, transcriptome sequencing, epigenome sequencing, or the like. A major premise of NGS is that different sequencing adapters need to be added to each of the two ends of a target sequence for tremendous different sequences, which is the so-called sequencing library preparation. In recent years, the single-cell sequencing technology has developed rapidly, and has led to important achievements in research fields such as reproduction, growth, differentiation, aging, and tumor research, but high experimental expenses and high-quality requirement of library are the key obstacles in front of researchers. Therefore, high-throughput, low-cost, and high-quality single-cell library preparation technologies and corresponding sequencing strategies have promising prospects.

The traditional single-cell genome sequencing technology and bulk-cell genome sequencing technology are basically the same in terms of preparation of a sequencing library, which involves steps such as DNA fragmentation, adapter addition, and polymerase chain reaction (PCR). The difference is that, in order to ensure a sufficient amount of start DNA to allow fragmentation of the genomic sequence through ultrasonic treatment or enzyme digestion, the single-cell sequencing requires pre-amplification by a special single-cell genome amplification method, such as MDA, MALBAC, and DOP-PCR, because a given cell has only 2 copies for any of the DNA sequences. The above process increases the cost of single-cell genome sequencing and the detection bias, and causes inaccurate copy number detection. Therefore, from acquisition of single cells to preparation of an actual sequencing library, the single-cell genome sequencing technology currently involves cumbersome steps, requires a large amount of reagent consumables, and is time-consuming, labor-intensive, and costly.

The single-cell genome sequencing mainly includes copy number variation (CNV) sequencing and single nucleotide variant (SNV) sequencing (the SNV is not involved in the present application). Low-throughput (generally, a library is constructed independently for each single cell) single-cell genome sequencing is expensive, time-consuming, and labor-intensive. High-throughput single-cell genome sequencing emerging in recent years greatly improve the process efficiency. Although the high-throughput single-cell genome sequencing has huge potential values in some research fields such as tumor research, the high-throughput single-cell genome sequencing is prohibitive due to its high cost and faces many practical limitations in some important clinical testing applications, including: (1) The number of single cells that requires sequencing in clinical practice is usually not large. For example, the preimplantation genetic test (PGT) requires only 8 to 13 trophoblast cells or even requires only 3 to 5 cells. There are generally only 3 to 23 circulating tumor cells (CTCs) in 2 mL of routine blood of a patient, and medium throughput generally refers only tens to hundreds of single cells in a test. (2) It is impossible to accurately trace an origin of a specified single cell. In the current high-throughput technology, single-cell library construction using barcode sequences accurately label a single cell different from other single cells at the early stage during library construction, however during bioinformatics analysis when output sequencing data is split into different single-cell data, it is impossible to accurately determine a pre-designated single cell to which the specified data belongs to. (3) The cost is high, which is reflected in library construction and sequencing. The cost of single-cell copy number variation (scCNV) sequencing (scCNV-seq) mainly lies in library construction while the sequencing cost alone is relatively low because only a shallow sequencing is required. In contrast to scCNV sequencing, both library construction and sequencing of SNV sequencing are expensive (no SNV innovation is involved in the present application). (4) A specialized expensive device is required for construction of a single-cell copy number library in high-throughput fashion.

At present, there is no desired technology available allowing for construction of a single-cell copy number library at a single-cell level on medium-throughput scale and separately labeling each specified cell at a very early stage, followed by sequencing (MT-scCNV-seq), which meanwhile should meet the requirement being fast, economical, efficient, and suitable for clinical application.

### SUMMARY

An objective of the present application is to overcome the shortcomings of the prior methods and provide a low-cost and high-efficiency MT-scCNV-seq method based on Tn5 transposase and specialized double-stranded oligonucleotoides or called adapters built from two oligonucleotides or called primers (the final complex is called Tn5 transposome), which is hereinafter referred to as MT-scCNV-seq (CNV: copy number variation, indicating CNVs of chromosomal or subchromosomal regions or DNA fragments; sc: single cell; and MT: medium throughput).

Medium throughput (MT) is provided merely relative to high throughput (HT) and low throughput (LT) of single-cell sequencing. HT of single-cell sequencing now refers to the simultaneous parallel operation of thousands of cells or more in an operating program, but the simultaneous parallel operation of hundreds of cells or even dozens of cells is sometimes considered as HT as well. LT refers to the independent construction of a library for each single cell. The MT method described here enables parallel scCNV-seq of several to hundreds of accurately-labeled single cells in a program, but it may treat thousands of single cells through combination of a plurality of programs; this method may also be adjusted and incorporated with a microfluidic system or computer-controlled robotic system to analyze hundreds to many thousands of single cells in parallel. Thus the method are also possibly to be classified as HT technology. However, in order to highlight the characteristics of the sequencing method of the present application, the sequencing method of the present application is named MT-scCNV-seq.

As one of the latest technologies for single-cell sequencing, scCNV-seq is a powerful tool for research on tumor heterogeneity and evolution, tumor biomarkers, reproductive health (detection of genetic disorders at embryo or fetal stage), drug screening, disease pathological mechanism, or the like. The current scCNV-seq technologies of LT are generally based on an independent single-cell whole genome amplification (WGA) for each cell, followed by independent library construction and sequencing of the amplified DNA, which lead to low efficiency in both cost and time, plus additional bias introduced during WGA. Although some HT scCNV-seq technologies have been reported in recent years, these HT scCNV-seq technologies require a huge number of loaded single cells, genome pre-amplification (preWGA) or depends on a microfluidic chip and a special sequencing scheme (not a conventional sequencing scheme, such as requiring multiple rounds of sequencing), particularly with random labeling of single cells, and thus they are not suitable for clinical sample testing in terms of time, efficiency, and traceability. Therefore, these methods do not have a wide applications, none for clinical application.

The MT-scCNV-seq of the present application is based on a set of oligonucleotides that are innovatively designed to build two double-stranded oligonucleotides or adapters for binding to Tn5 transposase, and the final complex is called Tn5 transposome. When the sequencing libraries for single cells ars constructed, a cell-specific barcode sequence is randomly inserted into the genome, and tagment the genome, of a given cell, while a different barcode is incorporated into the genome of another single cell, and so on. Then the labeling reactions of multiple single cells are pooled. The pooled mixture with multiple single cells are then PCR amplified, and sequencing library construction is conducted with a microreaction system in a single tube (PCR tube) with a batch index sequence. The batch indexes enable multiplex-library sequencing in a lane.

The Core innovations include: One-step direct construction of a pooled library of multiple single cells is applied, instead of the current independent amplification and independent library construction for each single cell; accurately labeling of the single cells (therefore tracable from a cell to the sequencing data, and from the sequencing data to the original cell) is used, instead of random single cell labeling (untraceable); this library is compatible to the public NGS platform, no unique or specific program is required on any sequencing platform. This innovation greatly improves the efficiency and quality over the current methods, and enables the MT-scCNV-seq fulfilling the requirements of clinical laboratories.

The present application adopts the following technical solutions:

A method for constructing an MT-scCNV-seq library, including:
providing sorted single cells;
independently lysing each single cell to fully expose a genomic DNA (gDNA) in the single cells;
tagmenting the gDNA and conducting sample-specific DNA labeling to obtain the whole set of fragmented gDNAs labeled with a cell-specific barcode in a given cell, while each cell has a different barcode; and
pooling the labeled barcoded fragmented gDNAs of a plurality of single cells (broadly speaking: samples) to collectively construct a MT-scCNV-seq library for subsequent sequencing,
wherein Tn5 transposome is used to tagment the gDNA in the single cell and label each gDNA fragments with a barcode; and
further, after NGS is completed with the constructed sequencing library, data output is analyzed by a relevant program (such as: baslan 2012, Gingo and Hmmcopy), wherein the output data collectively obtained are disaggregated to different cells, and the data for each single cell are separately traced to the original cell identity, and then determine the DNA copy number profile over the whole genome of each cell.

It should be noted that tagmenting gDNA and conducting sample-specific DNA labeling is intended to acquire fragmented gDNAs and label all fragments of gDNA of each cell with a cell-specific barcode.

Preferably, the sequencing library for single-cell genomic sequencing is compatible with Illumina NGS system and other NGS systems.

Preferably, the Tn5 transposome includes Tn5 transposase and two double strands of oligonucleotides, while one double strand of oligonucleotides Tn5P5 adapter is annealed from primer A and primer C, and the other double strand of oligonucleotides Tn5P7 adapter is annealed from primer B and primer C;

The primer A includes a cell barcode labeling sequence consisting of 3 to 23 single nucleotides, P5 PCR handle sequence, and reverse mosaic end (ME) sequence;

The primer B includes P7 PCR handle sequence and the reverse ME sequence; and

The primer C is an oligonucleotide with a phosphorylated 5′ terminus, and is partially complementary to each of the primer A and the primer B.

Or, the Tn5 transposome includes Tn5 transposase and two double strands of oligonucleotides, while one double strand of oligonucleotides Tn5P5 adapter is annealed from primer A and primer C, and the other double strand of oligonucleotides Tn5P7 adapter is annealed from primer B and primer C;

The primer A includes a cell barcode labeling sequence consisting of 3 to 23 single nucleotides, P7 PCR handle sequence, and the reverse ME sequence;

The primer B includes P5 PCR handle sequence and the reverse ME sequence; and

The primer C is an oligonucleotide with a phosphorylated 5′ terminus, and is partially complementary to each of the primer A and the primer B.

The barcode in the primer A is preferably 9 to 18, 11 to 17, or 12 to 15 and more preferably 11 nucleotides in length.

Preferably, the primer A has the nucleotide sequence shown in SEQ ID NO: 1-48.

Preferably, the primer B has the nucleotide sequence shown in SEQ ID NO: 49.

Preferably, the primer C has the nucleotide sequence shown in SEQ ID NO: 50.

Preferably, the method further includes the following steps:
(1) adding multiple single cell each to a different independent single tube;
(2) lysing each single cell in its tube with a lysis buffer or protease;
(3) inactivating the protease and optionally purifying the lysate or diluting the lysate to eliminate any factor from inhibition on the subsequent reaction;
(4) using the Tn5 transposome to tagment the gDNA obtained after lysing the single cell, and adding a single-cell-specific barcode recognition sequence consisting of 3 to 23 single nucleotides to the gDNA;
(5) pooling fragmented gDNA samples of a plurality of single cells in a single tube, and purifying the fragmented gDNAs, and then concentrating the fragmented gDNAs;
(6) subjecting the concentrated gDNA samples in the single tube as a batch of cells (broadly: samples) to PCR amplification to construct a multi-sample library of this batch of single cells in parallel in the single tube, wherein PCR amplification primers that include a specific batch index sequence compatible with an NGS system are adopted for each batch of gDNA samples; and
(7) purifying the multi-sample library, and recovering an aimed range of DNA sizes for the multi-sample library, in which the library length are selected from 300 bp to 1000 bp, or any size range in between.

Preferably, in step (6), an anchor sequence and a cell barcode sequence are added to a 5′ terminus of each insert DNA fragment, and subsequently, when the DNA fragment is amplified, an amplification adapter sequence compatible with a NGS system is added to each of upstream and downstream primers for the amplification; and

MT-scCNV-seq library from 5′ terminus to 3′ terminus sequentially includes the P5 adapter sequence, the first index sequence, the first sequencing primer binding site, the cell barcode sequence, the anchor sequence, the insert DNA fragment, the second sequencing primer binding site, the second index sequence, and the P7 adapter sequence sequentially, and all amplified DNA fragments constitute an library compatible with the NGS sequencing system.

Preferably, the NGS system is an Illumina sequencing system or another sequencing system/platform.

Preferably, the cell barcode sequence is an oligonucleotide sequence with 3 to 23 nucleotides including 2 to 5 random nucleotides and 1 to 18 nucleotides constituting a barcode. There are preferably 3 random nucleotides; and there are preferably 3 to 15, more preferably 5 to 12, and most preferably 8 nucleotides for the barcode.

Preferably, the anchor sequence is 5′-AGATGTGTATAAGAGACAG-3′ (SEQ ID NO: 51).

Preferably, in the NGS library, a specific structure of the amplified DNA fragment is as follows: 5′-AATGATACGGCGACCACCGAGATCTACAC(SEQ ID NO: 54) (index1)TCGTCGGCAGCGTCAGATGTGTATAAGAGACAG (SEQ ID NO: 52)(NNN+barcode consisting of M bases)AGATGTGTATAAGAGACAG (SEQ ID NO: 51)-TARGET-CTGTCTCTTATACACATCTCCGAGCCCACGAGAC(SEQ ID NO: 55)(index2)ATCTCGTATGCCGTCTTCTGCTTG (SEQ ID NO: 56)-3′, wherein “TARGET” represents the DNA fragment to be tested, “N” represents any one selected from the group consisting of bases A, T, C, and G, and “M” is 1 to 18.

Preferably, the single cell is replaced with a micro-bulk of cells, and the micro-bulk cells refers to 2 to 50, 50 to 100, 100 to 200, 200 to 500, or 500 to 1000 cells.

Preferably, the single cell is replaced with gDNA, and an amount of the gDNA is 1 pg to 1 μg.

Preferably, in step (2), the sorted single cell or micro-bulk cells in the tube is/are lysed with a detergent-containing lysis buffer or a Zymo genomic lysis buffer or a Qiagen protease.

Preferably, the relevant program and method for analyzing the data output to determine the copy number includes analysis software, an algorithm, a database, a website, and a visualization scheme.

The present application also provides a method of basic research, clinical screening, diagnosis, treatment, and drug research and development for a tumor, including:
constructing a copy number sequencing library of single cells or micro-bulk cells or corresponding gDNAs of a target subject; and
sequencing the copy number sequencing library,
wherein the copy number sequencing library is constructed by the method described above; and
the single cells or micro-bulk cells of the target subject are derived from a solid tumor tissue, a leukemia sample, CTCs, a minimal residual disease (MRD) sample, a fine needle aspiration biopsy sample, a hydrothorax sample, a hydroperitoneum sample, a urine sample, a vaginal sample, a cervical sample, or a cerebrospinal fluid, or the single cells of the target subject are single cells from a subject of another liquid biopsy or a surgical treatment.

The present application also provides a method of basic research, clinical screening, diagnosis, treatment, and drug research and development for fertility and reproduction genetics, including:
constructing CNV-seq libraries of single cells or micro-bulk cells or corresponding gDNAs of a target subject; and
sequencing the copy number sequencing library,
wherein the copy number sequencing library is constructed by the method described above; and
the single cells or micro-bulk cells of the target subject are derived from a non-invasive prenatal test (NIPT) subject, a prenatal diagnosis (PD) subject, a PGT subject, or a genetic test of miscarriage product subject.

The present application also provides a hardware system for HT gDNA copy number variation sequencing, including: a microfluidic chip, or a cell recognition, enrichment, and sorting system, or an automated liquid delivering system, and a computer software program configured to implement the hardware system, where the microfluidic chip or the cell recognition, enrichment, and sorting system is configured to sort and acquire target single cells and construct a sequencing library, and the sequencing library is constructed by the method described above.

The present application has the following beneficial effects:

The present method reaches to an MT level or even an HT level depending on requirements of an experiment. It is mainly reflected in that a sample is prepared into single-cell suspension according to actual conditions, and then single cells are captured and isolated by a 1 μL to 10 μL pipette with a filter cartridge or other alternative sorting, capturing and delivering system; or when an HT is required, a single-cell sorting system such as a FACS (Fluorescence-activated cell sorting, or Flow Cytometry, and the like) on the market is adopted for the sorting and delivery. According to the method of the present application, in many cases only an ordinary 96-well plate, or an 8-tube, or a 12-tube strip is required for the cell delivery, and there is no need for a microfluidic chip and a special water-in-oil magnetic bead or microwell system specifically required by a single-cell sequencing company. When a 96-well plate or an 8-tube strip or a 12-tube strip includes a single cell per well (system: about 1 μL); a core of the method of the present application is tagmentation with a self-designed barcode-containing Tn5 transposome (that is, a recognizable sequence is added). Moreover, an optimized reaction system undergoes gDNA fragmentation and adapter addition reactions in a 5 μL or down to nanoliter volume of reaction solution environment, so that the gDNA of each single cell is tagmentated randomly, and all fragments of the cells are labeled with the same barcode. Subsequently, a plurality of single cells are directly pooled and purified. In this way, a sequencing library is then constructed through direct PCR amplification, which directly builds the defined different sequences (sequencing primers, indexes, anchoring tag, and so on, and the associated matching sequences) on the two termini of the libraries for the aimed NGS platform with a batch index, without pre-amplification of each single cells. Due to a PCR suppression effect, when a transposome has the same DNA sequences at two termini due to irresistible reasons, a hairpin structure is formed during the denaturing and reannealing step of the PCR amplification to inhibit the amplification, which efficiently reduces the amplification efficiency of the described unwanted constructs (with the exact same two termini on a construct). and leads to PCR products only containing different termini right for NGS sequencing. When multiple batch of single cells (or broadly: samples) are processed due to the cells number being analyzed are large, different commercially-available indexes may be used in different batches of cells in this amplification step. These designs have been successfully tested, fitting the commercially-available kits (such as Vazyme, Illumina or the like) and the Illumina NGS platform.

Therefore, the method of the present application allows easy and fast construction of a MT-scCNV-seq library of tens to hundreds of cells in a row. The present application also provides a cutting-edge technology for research and clinical applications of tumor single cells such as CTCs, reproduction genetic testing such as PGT and NIPT, and single-cell PD for a liquid biopsy in a clinical sample and early diagnosis of other diseases related to CNVs (or CNAs: copy number alternations), and promotes the development of the entire biomedicine.

### BRIEF DESCRIPTION OF THE DRAWINGS

FIG. 1 is a schematic flow chart of the method for constructing an MT-scCNV-seq library in the present application;

FIG. 2A and FIG. 2B each are a schematic diagram illustrating an assembly of Tn5 transposase with two double strands of oligonucleotides, where in FIG. 2A is a schematic diagram illustrating an assembly of oligonucleotide sequences A, B, and C with the Tn5 transposase to produce the Tn5 transposome; and FIG. 2B is a schematic diagram illustrating an assembly of Tn5P5 adapter is annealed from primer A and primer C; and Tn5P7 adapter is annealed from primer B and primer C. The Tn5 transposase is incorporated with Tn5P5 adapter and Tn5P7 adapter, producing Tn5 transposome;

FIG. 3 is a schematic diagram of single-cell capture, wherein the small black dot inside the circle refers to a captured single cell;

FIG. 4 is a schematic structural diagram of a sequencing library obtained after PCR amplification and purification, wherein P5 represents the P5 adapter sequence; Index1 represents an index sequence for recognizing a sample; Rd1 SP represents the first sequencing primer-binding sequence for one terminus of double-terminal sequencing; BC represents the barcode sequence for recognizing a single cell; ME represents an anchor sequence for locating the barcode sequence and the same as ME sequence; DNA insert represents a fragment to be sequenced; Rd2 SP represents a sequencing primer-binding sequence for the other terminus of double-terminal sequencing; Index2 represents a tag sequence at a P7 terminus; and P7 represents a P7 adapter sequence;

FIG. 5 shows schematic E-Gel analysis of an MT-scCNV-seq library of 16 cells in each of 4 batches of K562 cells, and gel extraction (300 bp to 500 bp);

FIG. 6 shows schematic E-Gel analysis of an MT-scCNV-seq library for a Jurkat cell line (n=16) and a normal human peripheral blood mononuclear cell (PBMC) (n=16), and gel extraction (300 bp to 500 bp);

FIG. 7 shows schematic E-Gel analysis of an MT-scCNV-seq library for a GM12878 cell line (48 single cells are pooled for library construction), and gel extraction (300 bp to 500 bp);

FIG. 8 shows detection results of fragments in MT-scCNV-seq library constructed for a K562 cell line by Agilent 2100, which has been smoothed, wherein the kurtosis is 300 bp to 800 bp, which complies with the standards of NGS; and the right rectangle shows a DNA electrophoresis pattern, wherein the darker the gray scale, the more concentrated the DNA in this region;

FIG. 9 shows detection results of fragments in scCNV-seq libraries constructed for a normal control and a Jurkat cell line by Agilent 2100, which has been smoothed, wherein the kurtosis is 300 bp to 800 bp, which complies with the standards of NGS; the normal control refers to a normal human PBMC, and 48 single cells are pooled for library construction of the normal human PBMC; 48 single cells are pooled for library construction of the Jurkat cell line; and the right rectangle shows a DNA electrophoresis pattern, wherein the darker the gray scale, the more concentrated the DNA in this region; and

FIG. 10 shows detection results of fragments in MT-scCNV-seq library constructed for a GM12878 cell line by an Agilent 2100 nucleic acid analyzer, which has been smoothed, wherein the kurtosis is 300 bp to 800 bp, which complies with the standards of NGS; and 48 single cells are pooled for library construction of the GM12878 cell line.

### DETAILED DESCRIPTION

In the present application, the term “LT” library construction refers to a library construction type in which a sequencing library is independently constructed for each single cell during construction of a NGS sequencing library.

The term “MT” library construction refers to a library construction type that supports the simultaneous parallel construction of sequencing libraries of several cells, tens of cells, or even hundreds of or more cells (generally, 2 to 500 cells and preferably 10 to 100 cells) in an operating program during construction of a NGS sequencing library.

The term “HT” library construction refers to a library construction type in which sequencing libraries of hundreds of or tens of thousands of cells (generally, 100 or more to tens of thousands of single cells, and preferably, 1,000 to more than 10,000 single cells) are simultaneously constructed in parallel in an operating program.

The term “copy number” refers to a number of copies of a specified gene or a specified DNA sequence long or short. Human is a diploid organism, in which an allele normally is 2 copies.

The term “CNV” refers to an increase or a decrease of the copy number of a genomic DNA fragment or sequence with a length generally of 50 bp or more, up to megabase or a whole chromosome, usually resulting from a genome rearrangement. CNV is mainly manifested as chromosomal microdeletion and microduplication at a submicroscopic level, and also is called chromosomal CNV and DNA CNV. CNV is one of the important genetic pathogenic factors for human diseases. For example, in general, each gene and each chromosome fragment of a normal human somatic cell is a diploid (2 copies), and if the copies increase or decrease, there is a CNV. Trisomy 21 is a characteristic chromosomal CNV of Down syndrome.

The term “scCNV-seq library” refers to a NGS sequencing library constructed for application to NGS sequencing platform to detect the genomic copy number at single-cell level.

The term “sorting” refers to a process of distinguishing different cell types based on parameters such as size, physical characteristics, and especially cell surface antigen expression (markers) of the cells to obtain the target cells. “Delivery” actually refers to placement of a single cell in a specific reaction tube or a reaction well.

The term “capture, isolation, and delivery of single cells” refers to selection, enrichment, and acquisition of single cells on a medium or a tissue and transfer of the single cells to a new reaction environment by a specific method.

The term “purification” refers to separation of nucleic acid from other macromolecular substances such as proteins, polysaccharides, and fats, and substrates left after a reaction, to exclude molecular impurities and obtain high-quality target nucleic acid.

The term “amplification” refers to 1) a selective increase in a number of copies of one or more genes or chromosome fragments in an organism, or 2) DNA amplification conducted in a laboratory.

The term “DNA amplification”, also known as “DNA fragment amplification”, refers to a process of increasing a number of copies of a specific DNA sequence through replication. If occurring at a large scale (chromosomal or subchromosomal level), the DNA amplification may be called “chromosomal duplication or amplification” or “chromosomal segmental duplication or amplification”. The DNA amplification may occur in vivo or in vitro. The in vitro amplification is conducted by a PCR technique or another specific technique. The “cell expansion” refers to proliferation of cells through cultivation.

The term “upstream and downstream primers” refers to an upstream primer and a downstream primer, wherein the upstream primer is also known as a forward primer and the downstream primer is also known as a reverse primer. DNA replication is always conducted from 5′ terminus to 3′ terminus, where the upstream primer is close to the 5′ terminus and the downstream primer is close to the 3′ terminus.

The term “WGA” (Whole genome amplification) refers to an in vitro experimental technique for amplification of DNA of the entire genome described above. The WGA is intended to faithfully amplify DNA of the entire genome (rather than a specific DNA fragment or gene) proportionally.

The term “cell lysis” refers to release of cell contents and nucleic acids by changing the permeability of a cell, and dissolving cell membrane, nuclear membrane and other macromolecules.

The term “fragmentation” (wherein fragmentation that not only fragments DNA but also labels DNA through Tn5 transposome is called tagmentation) refers to fragmentation or cleavage of a large nucleic acid into small fragments by a physical method (an ultrasonic treatment) or an enzymatic method (a non-specific endonuclease or transposase).

The term “sequencing library” refers to an entire set of DNA or an entire set of cDNA fragments transcribed from DNA, RNA, or a sum of target sequences of a specific type, in which an adapter sequence corresponding to a particular sequencing platform is included at each terminus. In other words, the term “sequencing library” refers to a molecular clone fragment obtained after a specific adapter is linked to each terminus of a DNA fragment, including a sequence that is recognized by primer clusters in a flow cell of a NGS sequencer and a generic sequence that is used to amplify the inserted DNA.

The term “adapter” refers to a paired oligonucleotides that are provided to link a target fragment to be sequenced. An adapter includes a specific sequence required for sequencing and later analysis, for example, an anchor sequence that is complementary to a cluster sequence generated in an NGS flow cell; a sequencing primer sequence that provides a sequencing primer binding site to initiate sequencing; an amplification primer sequence or its complementary sequence that is provided for library amplification; and a cell barcode sequence and an index sequence that provide cell labeling and library batch labeling, respectively.

The term “Tn5 transposase” refers to a wide class of proteins, actually enzymes, derived from bacteria. Tn5 transposome is constructed by the Tn5 transposase and its associated oligonucleotides, which tagment target genomic DNA and insert a part of the oligonucleotide of the transposome into the target sequence under specified conditions, so that DNA fragments for library construction are directly amplified by PCR, and barcode the PCR product on the same time.

The term “Tn5 transposome” refers to an action system of a transposase complex (transposome) produced with two molecules of a Tn5 transposase and two double strands of oligonucleotides, which allows insertion of transposome DNA into a target sequence while allowing fragmentation of the target DNA at a specific temperature in a reaction buffer.

The term “anchor sequence” in the present application refers the following two situations: (1) When involving Tn5 transposome system, the anchor sequence refers to the ME sequence for Tn5 transposase, generally 19 bp, which is also the Tn5 transposase binding sequencing site. (2) When involving an adapter sequence of a sequencing library, the anchor sequence is a sequence complementary to a cluster sequence generated on a NGS flow cell. The two sequences themselves may be similar to each other or overlap with each other, or different from each other.

The term “reverse ME sequence” refers to a reverse complementary sequence for a ME sequence of Tn5 transposase, which complements to a ME sequence and forms a DNA double-stranded structure during preparation of a transposome system.

The term “P5 adapter sequence” refers to a sequence on an Illumina NGS platform that allows library binding and is complementary to a cluster generated in a Flow Cell, wherein an adapter defined as complementary to a P5 cluster sequence in a Flow Cell is called a P5 adapter.

The term “P7 adapter sequence” refers to a sequence on an Illumina NGS platform that allows library binding and is complementary to a cluster generated in a Flow Cell, wherein an adapter defined as complementary to a P7 cluster sequence in a Flow cell is called a P7 adapter.

The term “Tn5P5 adapter” refers to an double-strands of oligonucleotides, being used to bind to Tn5 transposase and to construct the active Tn5 transposome. Beyond the double strand portion, The “Tn5P5 adapter” contains a single strand portion, i.e the “P5 PCR handle”. This “Tn5P5 adapter” is developed in this method MT-scCNV-seq, and is different from the “P5 adapter” widely used in NGS sequencing.

The term “P5 PCR handle” refers to the oligonucleotide, which is a part of the “P5 adapter sequence”, used in “Tn5P5 adapter” for priming of the P5 primer to enable PCR amplification for the library construction.

The term “Tn5P7 adapter” refers to an double-strands of oligonucleotides, being used to bind to Tn5 transposase and to construct the active Tn5 transposome. Beyond the double strand portion, it contains a single strand portion, i.e the “P7 PCR handle”. This “Tn5P7 adapter” is developed in this method MT-scCNV-seq, and is different from the “P7 adapter” widely used in NGS sequencing.

The term “P7 PCR handle” refers to the oligonucleotide, which is a part of the “P7 adapter sequence”, used in “Tn5P7 adapter” for priming of the P7 primer to enable PCR amplification for the library construction.

The terms “the first index sequence” and “the second index sequence” refer to two index tag sequences for distinguishing samples, which allow single sequencing or pooling of a plurality of samples (or single cells) in a single Flow Cell channel. In the present application, a tag sequence of the P5 adapter sequence is called the first index sequence; and a tag sequence of the P7 adapter sequence is called the second index sequence.

The term “the first sequencing primer binding site” refers to a site on an Illumina sequencing platform or another general-purpose sequencing platform that allows binding of a sequencing primer to an oligonucleotide sequence close to a P5 terminus during sequencing, or a corresponding site on another sequencing platform.

The term “the second sequencing primer binding site” refers to a site on an Illumina sequencing platform or another general-purpose sequencing platform that allows binding of a sequencing primer to an oligonucleotide sequence close to a P7 terminus during sequencing, or a corresponding site on another sequencing platform.

The term “precise labeling with a barcode” refers to a use of an oligonucleotide (a molecular barcode, composed of a combination of multiple nucleotides) with a specified length to accurately label different related molecules, including the following different uses: (1) a cell barcode for cell-specific barcode labeling; (2) broadly including an index sequence; and (3) an unique molecular index (UMI), which is also a molecular barcode, used to label an original DNA or RNA molecule to distinguish the original molecule from a molecule amplified later, thereby allowing correction of a copy number deviation of a sequencing result.

In the present application, the term “barcode”, namely, refers to a cell barcode, which is a common ID for all DNA fragments specific to a single cell. If micro-bulk cells (as an independent sample) are subjected to copy number sequencing, the barcode may also refer to a specific barcode or ID of an independent sample composed of a set of cells collectively.

In the present application, the term “barcode recognition sequence” refers to a combination sequence of the barcode and 3 random nucleotides in front of the barcode. The barcode recognition sequence is provided to recognize a specific single cell during analysis of sequencing data. The 3 random nucleotides are provided to meet the randomness requirement (because the barcode itself is not a random sequence) of the sequencing system (an Illumina NGS system), which is usually required.

The term “cell barcode sequence” refers to a combination sequence consisting of the 3 random nucleotides (“barcode recognition sequence”) and the barcode. The cell barcode sequence is provided to label all fragments sequenced of a given single cell.

In the present application, the term “recognizable sequence” refers to a known artificial sequence that is recognizable during analysis. The barcode and the index are of recognizable sequences.

The term “index”, also known as “index sequence”, refers to an oligonucleotide sequence to distinguish a specified library from another library during NHS sequencing. The index allow single sequencing or pooling of a plurality of samples in a single Flow Cell channel, and in the latter case, data is split according to a specified index of a specified library.

The term “conventional NGS platform” refers to an NGS sequencing platform commonly used in the industry, and mainly refers to an Illumina-based sequencing platform. However, the conventional NGS platform also includes the latest released sequencing platform such as but not limited to an MGI sequencing system of BGI.

In the present application, the term “amplification adapter sequence” refers to an adapter sequence on which amplification relies. The “amplification” here refers to DNA amplification during library construction. The amplification adapter sequence may also refer to an adapter sequence for library amplification that is recognized and complemented by an oligonucleotide cluster on Flow Cell of an NGS sequencing platform.

The term “sequencing lane” refers to a flow slot on a sequencing chip. A sequencing library and a reagent are in the slot; the scanning of a sequencing signal is conducted according to a subunit tile on a lane; and a flow cell has a single or a plurality of lanes.

The term “multiplex-library sequencing in a lane” (a sample is sequenced in combination with other samples; a sample is sequenced in combination with other sample/s in a lane instead of being sequenced independently in a whole lane) refers to sequencing of a combination of sequencing libraries derived from different sources and different types in a same lane at one time relative to “single library sequencing in a lane”.

The term “reproductive health” refers to an industry field of research on physical, mental, and social health states involved by a reproductive system and functions thereof. In the present application, the reproductive health mainly refers to reproduction-related clinical genetic health of the embryo and the fetal in addition to the parents and its associated tests, including but not limited to pregestational test, genetic test of miscarriage product, PGT, PD, and NIPT.

The term “massive health” refers to prevention-based health management, which is summarized as various production and service fields closely related to human health, such as medical services, medicine and health care products, nutrition and health care products, medical health care instruments, leisure health care services, and health consultation management.

The term “target cell/s” refers to a target cell, a single cell, or a bulk of cells detected, processed, or studied in an experiment.

Regarding the term “decoding”, the decoding of sequencing data refers to splitting (data disaggregation) and identification of a specific sequence data with regard to the originally processed cells (samples), including identification and splitting of data derived from different tag sequences, different cell sources, and different samples. Decoding is often conducted according to various barcodes and indexes.

In the present application, the term “pre-amplification” (pre-whole genome amplification, preWGA) indicates that conventional single-cell DNA sequencing requires WGA for a genome of a sample (a bulk sample or a single cell) to increase to a relatively higher level of quantity that is qualified for processing, and then a sequencing library is independently constructed. Ideally preWGA is expected to be unbiased and faithful, ie all sequences should be amplified on the same ratio, without ratio distortion, and the sequence should be exactly the same as the original template without sequence mutation. However, in real experiment, every preWGA introduces bias and distortion more or less. In addition, a library without a complete adapter is sometimes first subjected to one-step amplification, which is called pre-amplification; and a pre-amplification product is purified and then subjected to a second round of amplification to add a complete adapter sequence.

The term “transcriptome sequencing” refers to sequencing of a cDNA library transcribed from all RNAs in a tissue or a cell or a bulk of cells by an NGS technology and investigation of gene transcription and transcription regulation of the target samples.

The term “microfluidic chip” is a technology mainly characterized by manipulation of a fluid in a micro-scale space. At present, the mainstream microfluidic chip refers to a chip on which basic operation units such as sample preparation, reaction, isolation, detection, cell cultivation, sorting, and lysis are integrated, and mainly includes a micro-well system and a droplet system.

The term “water-in-oil magnetic beads” refers to water-in-oil droplets formed by cells and magnetic beads after cells are shunted into a water-in-oil emulsion to form independent reaction chambers (cells, magnetic beads, and reaction reagents are in oil droplets). For example, in 10× Genomics single-cell transcriptome sequencing, a single magnetic bead and a cell are wrapped by a droplet to form an independent reaction space.

The term “micro-well system” refers to independent reaction chambers formed by shunting cells into a micro-well array. For example, in BD single-cell transcriptome sequencing, based on microwells, hundreds to thousands of single cells are captured and labeled with a barcode, and then genome and proteome information is analyzed.

The term “PCR suppression effect” refers to the fact that, at a low primer concentration, two termini of a non-specific product strand with a small length (including a primer dimer) are easily paired with each other to form a stem-loop structure to prevent primer binding, thereby strongly inhibiting PCR amplification.

The term “hairpin structure” refers to a structure in which complementary bases are paired with each other through self-folding due to a double-symmetry region on a single-stranded DNA or RNA molecule to form a local region with a hydrogen-bonded double-stranded structure.

The term “micro-bulk cells” usually refers to a sample including 2 to 5,000 cells, preferably 2 to 1,000 cells, and more preferably 5 to 100 cells.

### Embodiments

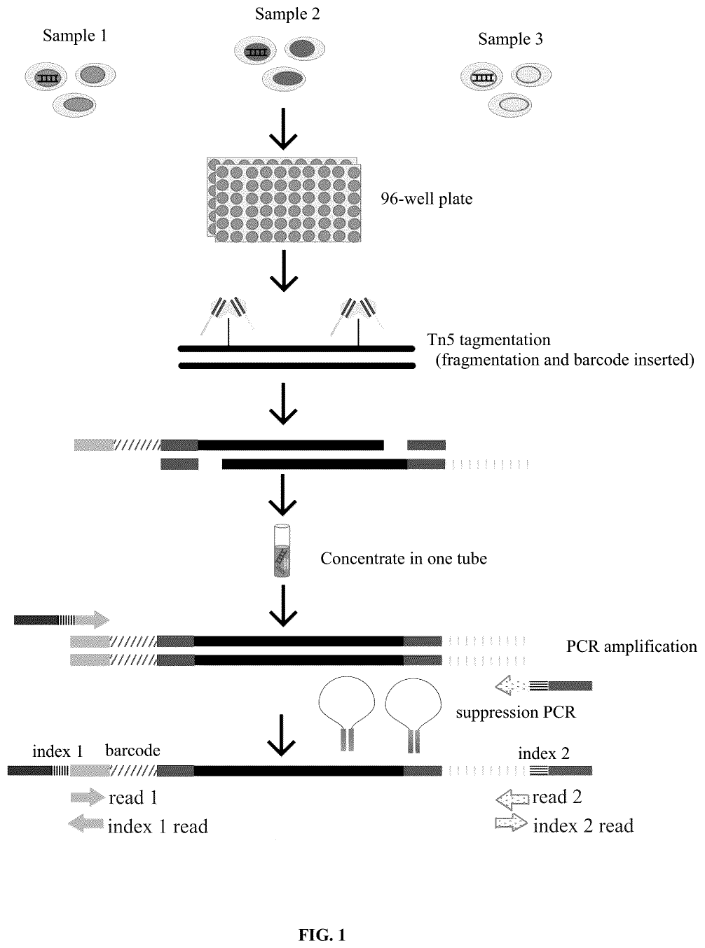

The method for constructing an MT-scCNV-seq library in the present application is shown in FIG. 1.

In some embodiments, a method for constructing an MT-scCNV-seq library and sequencing is provided, including: lysis of each sorted single cell in a multi-well plate or strip, cell lysis, and DNA tagmenting and library construction based on Tn5 transposome to obtain a genomic sequencing library for subsequent sequencing; and the method specifically includes the following steps:
1) sorting and capture of single cells: single cells are captured to a multi-well plate including but not limited to a 96-well or 384-well plate or a strip-tube including but not limited to an 8-strip or 12-strip tube;
2) lysis of single cells: each sorted single cell in a tube is lysed with a Zymo genomic lysis buffer or a Qiagen lysis buffer or a protease K or other lysis buffer based on one or more detergents to fully expose gDNA;
3) reaction treatment: the lyase in a single-cell lysate is inactivated, and the gDNA sample is purified or diluted to remove inhibition of the above lysis reagent on a subsequent reaction;
4) library construction with Tn5 transposome system: based on Tn5 transposome system, the single-cell gDNA is tragmented, and a cell-specific barcode recognition sequence consisting of N (3 to 23) single nucleotides is added to all DNA fragments of each cell;
5) fragmented gDNA samples of a plurality of single cells are pooled in a single tube, and the fragmented gDNA samples are purified and concentrated;
6) In parallel construction of a multi-sample library in a single tube: the concentrated gDNA samples in the single tube is subjected as a batch of samples to PCR amplification to construct a multi-sample library of this batch, wherein PCR amplification primers that include a specific batch of index sequences and are compatible with an NGS system are adopted for each batch of gDNA samples;
7) the multi-sample library is purified, and a DNA length of the multi-sample library is selected;
8) the multi-sample library is subjected to NGS, and the output sequencing data is subjected to single-cell-specific decoding; and
9) the sequencing data is subjected to downstream analysis.

In some embodiments, step 4) includes: Tn5 transposome is added to a single-cell gDNA solution to allow a reaction, and then an enzyme inhibitor is added to completely terminate the fragmentation reaction with Tn5 transposome and the enzymatic activity of the Tn5 transposase.

In some embodiments, in step 1), the single cells may be sorted by a flow cell sorter, or another alternative sorting device, or a cell type-specific enrichment device, including but not limited to a cellenONE or Namocell single-cell sorter.

In some embodiments, in step 2), the single cells are lysed with a Zymo genomic lysis buffer (Cat. No. D3004-1-50).

In some embodiments, in step 2), the single cells are lysed with a Qiagen protease (Cat. No. 19155/19157); and after the lysis is completed, a cell lyase is inactivated by heating.

In some embodiments, in step 3), DNA is purified with an AMPure XP (Cat. No. A63881) magnetic bead or another magnetic bead capable of purifying DNA.

In some embodiments, in step 4), the library construction with the Tn5 transposome includes the following steps: Tn5 transposome is added to a single-cell DNA solution to allow a reaction, and then an enzyme inhibitor is added to completely terminate a fragmentation reaction of the Tn5 transposome and an enzymatic activity of the Tn5 transposase.

In some embodiments, the Tn5 transposome includes Tn5 transposase and two double strands of oligonucleotides, while one double strand oligo Tn5P5 adapter is annealed from primer A and primer C, and the other double strand oligo Tn5P7 adapter is annealed from primer B and primer C; the primer A includes a cell barcode labeling sequence consisting of 3 to 23 single nucleotides, the P5 PCR handle sequence, and the reverse mosaic end (ME) sequence; the primer B includes the P7 PCR handle sequence and the reverse ME sequence; and the primer C is an oligonucleotide with a phosphorylated 5′ terminus, and is partially complementary to each of the primer A and the primer B. In some embodiments, the primer A is the nucleotide sequence shown in SEQ ID NO: 1-48, the primer B is the nucleotide sequence shown in SEQ ID NO: 49, and the primer C is the nucleotide sequence shown in SEQ ID NO: 50.

In some embodiments, in step 6), a specially-designed sequencing library is constructed, wherein an anchor sequence and a cell barcode sequence are added to a 5′ terminus of each DNA fragment to be tested, and subsequently, when the DNA fragment to be tested is amplified, an amplification adapter sequence compatible with the sequencing system is added to each of upstream and downstream primers for the amplification; and an amplified DNA fragment includes in order: the P5 adapter sequence, the first index, the first sequencing primer binding site, the cell barcode, the anchor sequence, the DNA fragment to be tested, the second sequencing primer binding site, the second index, and P7 adapter sequence sequentially from 5′ terminus to 3′ terminus. And all amplified DNA fragments finally constitute an NGS library compatible with an Illumina sequencing system.

In some embodiments, the cell barcode sequence consists of 3 random nucleotides and a nucleotide sequence with a length of 8 bp; the anchor sequence is AGATGTGTATAAGAGACAG (SEQ ID NO: 51); the first sequencing primer binding site is TCGTCGGCAGCGTCAGATGTGTATAAGAGACAGAGATGTGTATAAGAGACAG (SEQ ID NO: 52); and the second sequencing primer binding site is GTCTCGTGGGCTCGAGATGTGTATAAGAGACAG (SEQ ID NO: 53).

In some embodiments, in the NGS library, a specific structure of the amplified DNA fragment is as follows: 5′-AATGATACGGCGACCACCGAGATCTACAC(SEQ ID NO: 54)(index1)TCGTCGGCAGCGTCAGATGTGTATAAGAGACAG (SEQ ID NO: 52)(NNN+barcode consisting of M bases)AGATGTGTATAAGAGACAG (SEQ ID NO: 51)-TARGET-CTGTCTCTTATACACATCTCCGAGCCCACGAGAC(SEQ ID NO: 55)(index2)ATCTCGTATGCCGTCTTCTGCTTG (SEQ ID NO: 56)-3′,

wherein “TARGET” represents a DNA fragment to be tested, “N” represents any one selected from the group consisting of 4 nucleotides A, T, C, and G, and “M” is 1 to 18.

Nucleotide sequences involved in the DNA fragment are numbered as follows:

(SEQ ID NO: 54)
5′-AATGATACGGCGACCACCGAGATCTACAC;
(SEQ ID NO: 52)
5′-TCGTCGGCAGCGTCAGATGTGTATAAGAGACAG;
(SEQ ID NO: 51)
5′-AGATGTGTATAAGAGACAG;
(SEQ ID NO: 55)
5′-CTGTCTCTTATACACATCTCCGAGCCCACGAGAC;
(SEQ ID NO: 56)
5′-ATCTCGTATGCCGTCTTCTGCTTG.

In some embodiments, the anchor sequence is a nucleic acid/DNA sequence for stably finding an insertion location of a recognizing sequence in later sequencing data; and the index sequence 1 and the index sequence 2 both are index sequences for labeling experimental batches.

In some embodiments, in step 7), the library is purified through agarose gel electrophoresis sorting, and DNA fragments of the library are selectively recovered; and the DNA length of the library is selected by magnetic beads.

In some embodiments, in step 8), specific steps for NGS are as follows: a plurality of single-cell gDNA libraries with different index sequences are pooled, and then subjected to bulk sequencing in a same sequencing lane or directly according to a data amount required on an NGS platform.

In some embodiments, according to actual needs of a data amount, fragment size selection is conducted, and then DNA purification and sequencing is conducted; or DNA purification is directly conducted without fragment size selection, and then sequencing is conducted.

In some embodiments, the single cell is replaced with a plurality of cells, and the plurality of cells refers to 2 to 50, 50 to 100, 100 to 200, 200 to 500, 500 to 1,000, or 1,000 to 10,000 cells.

In some embodiments, the single cell is replaced with gDNA, and an amount of the gDNA is 1 pg to 1 μg.

In some embodiments, the present application provides a specific method of basic research, clinical diagnosis, treatment, and drug RD for a tumor, including: a scCNV-seq library of a target subject is constructed; and the library is sequenced, wherein the scCNV-seq library is constructed by the method described above.

In some embodiments, single cells of the target subject are single cells from a tumor tissue, single cells from a circulating tumor cells (CTCs), single cells from an MRD patient, single cells from a fine needle aspiration biopsy, single cells from hydrothorax, single cells from hydroperitoneum, single cells from urine, single cells from a cerebrospinal fluid, or single cells of any liquid biopsy subject.

In some embodiments, the present application also provides a method of basic research, cancer diagnosis, treatment, and new drug RD, and fertility and reproduction genetics, including: a scCNV-seq library of a target subject is constructed and sequenced, wherein the scCNV-seq library is constructed by the method described above.

In some embodiments, single cells of the target subject are single cells of an NIPT subject, single cells of a PD subject, single cells of a PGT subject, or single cells of a genetic test of miscarriage product subject.

In some embodiments, the present application also provides a use of the library construction method in preparation of a test kit, an experimental device, or a detection system related to basic research, clinical diagnosis, treatment and drug development for tumors, also in reproduction genetics test, and massive health.

In order to concisely and clearly demonstrate the technical solutions, objectives, and advantages of the present application, the present application is further described in detail in combination with specific embodiments and accompanying drawings. Unless otherwise specified, a concentration of a solid or liquid reagent in the present application refers to a mass concentration. Unless otherwise specified, the reagents and materials in the present application are commercially available.

### Embodiments

I. Design of Oligonucleotides and Construction of Tn5 Transposome System

A set of oligonucleotides (primers) need to designed for assembly of Tn5 transposome. The first part of ME sequence for binding to the Tn5 transposase. The second part is full priming sequence for PCR priming for library construction and adapter addition. Therefore, 3 oligonucleotides (called primer A, primer B, and primer C) to form two complementary double-stranded structures. These 3 oligonucleotides need to be pre-annealed.

Thus, the oligonucleotides of the Tn5 transposase of the present application include primer A, primer B, and primer C; the primer A includes a cell barcode labeling sequence consisting of 3 to 23 single nucleotides, the P5 PCR handle sequence, and the reverse mosaic end (ME) sequence; the primer B includes the P7 PCR handle sequence and the reverse ME sequence; and the primer C is an oligonucleotide with a phosphorylated 5′ terminus, and is partially complementary to each of the primer A and the primer B. and the primer A has a nucleotide sequence shown in SEQ ID NO: 1-48, the primer B has a nucleotide sequence shown in SEQ ID NO: 49, and the primer C has a nucleotide sequence shown in SEQ ID NO: 50.

The Tn5P5 adapter is provided to match a PCR amplification sequence at a 5′ terminus on an Illumina sequencing platform, which facilitates the addition of an official tag sequence (index 1) and a sequencing adapter 1 through PCR after a plurality of samples are pooled; and the Tn5P7 adapter is provided to match a PCR amplification sequence at a 7′ terminus on an Illumina sequencing platform, which also facilitates the addition of an official tag sequence (index2) and a sequencing adapter 2 through PCR after a plurality of samples are pooled. In this way, a Barcode×Index combination is produced to enable MT single-cell sequencing, which reduces a cost (there is no need to pack all flow cells or lanes, and different samples are pooled for sequencing).

1. Preparation of Two Double Strands of Oligonucleotides for Tn5 Transposase:

(1) Pre-Annealing of Double Strands of Oligonucleotides:

a. Since the primer A could be partially complementary to the primer C and the primer B could be partially complementary to the primer C, before a library construction reaction, the primers A and C and the primers B and C each needed to be annealed to produce double-stranded structures, namely, Tn5P5 and Tn5P7 adapters.

b. The Tsingke Biotechnology Co., Ltd. was entrusted to synthesize the oligos. A TE Buffer was added according to a description system to allow a concentration of 100 μM.

c. An annealing reaction system was prepared with a 1.5 mL centrifuge tube according to the following system:

TABLE 1
Reaction system for the Tn5P5 adapter

| Reactant | Volume | |
|---|---|---|
| Primer A(100 μM) (SEQ ID NO: 1-48) | 4 | μL |
| Primer C(100 μM) (SEQ ID NO: 50) | 4 | μL |
| T4 Ligation Buffer | 4 | μL |
| dd H₂O | 8 | μL |
| Total volume | 20 | μL |

TABLE 2
Reaction system for the Tn5P7 adapter

| Reactant | Volume | |
|---|---|---|
| Primer B(100 μM) (SEQ ID NO: 49) | 4 | μL |
| Primer C(100 μM) (SEQ ID NO: 50) | 4 | μL |
| T4 Ligation Buffer | 4 | μL |
| dd H₂O | 8 | μL |
| Total volume | 20 | μL |

d. The 1.5 mL centrifuge tube was wrapped with a tin foil to facilitate uniform heating for a subsequent reaction.

e. The 1.5 mL centrifuge tube with the reaction system was transferred into a 94° C. water bath to allow a reaction for 2 min, then a temperature was gradually reduced to 80° C. within 10 min, and the centrifuge tube was transferred to a clean environment and naturally cooled to room temperature.

f. A nucleic acid product resulting from pre-annealing could be stored in a −20° C. refrigerator for a subsequent scCNV-seq library construction experiment.

2. Assembly of Tn5 Transposome (or Called Tn5 Transposase Complex)

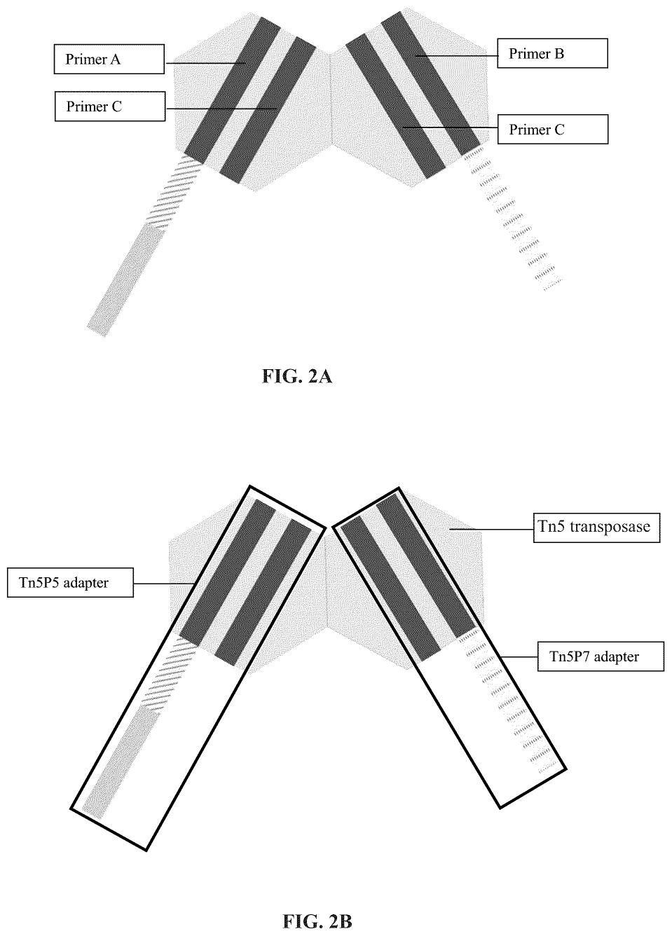

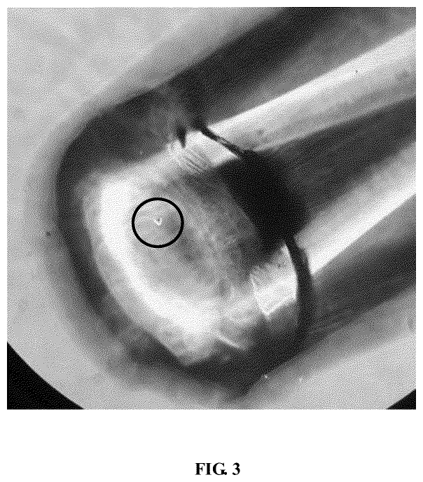

The Tn5 transposase recognizes double-stranded parts of the Tn5P5 and Tn5P7 adapters, and two different double-stranded nucleic acid products were then assembled with the Tn5 transposase to produce the Tn5 transposase complex that could be used for NGS, as shown in FIG. 2A and FIG. 2B.

Specific operations were as follows:

a. Tn5P5 and Tn5P7 adapter stock solutions were diluted 2-fold in a ratio of 1:1 to allow a final concentration of 101.64.

b. A reaction system was prepared according to the following system:

TABLE 3
Reaction system

| Reactant | Volume | |
|---|---|---|
| Tn5P5 adapter | 1 | μL |
| Tn5P7 adapter | 1 | μL |
| 10× TPS Buffer | 1 | μL |
| Tn5 Transposase(1 U/mL) | 2 | μL |
| ddH₂O | 5 | μL |
| | 10 | μL |

c. The reaction system was placed in a 37° C. metal bath to allow a reaction for 30 min.

d. A product of the reaction was a reaction enzyme with adapters, and could be used for the following scCNV-seq library construction or stored at −20° C.

II. Acquisition of Single Cells

1. Cell cultivation

A state of cells has a great impact on the method of the present application. If there is too much debris in a cell culture medium, the cell sorting under a microscope will be affected. If cells undergo malnutrition, a three-dimensional (3D) chromosomal structure or a chromatin structure of the whole cell may be affected to some degree, or the cells may die and produce cell debris. Specific steps of cell cultivation in this embodiment were as follows:

(1) Cell samples adopted in this embodiment included: a K562 cell line, a Jurkat cell line, and a GM12878 cell line. The K562 cell line was taken as an example.

(2) A K562 cell cryopreservation tube was placed in a 37° C. water bath for instant thawing.

(3) A thawed K562 cell suspension was centrifuged in a low-speed centrifuge at 800 rpm for 5 min.

(4) The cryopreservation tube with K562 cells was sprayed with 75% alcohol, and then placed in a clean bench for subsequent operations.

(5) A resulting supernatant was discarded by a 1,000 μL pipette, then 1,000 μL of phosphate buffered saline (PBS) was added to resuspend the cells, and a resulting mixture was repeatedly pipetted up and down for thorough mixing to obtain a cell suspension.

(6) The cell suspension was centrifuged in a low-speed centrifuge at 800 rpm for 4 min.

(7) A resulting supernatant was discarded, and the cells were resuspended with 1,000 μL of a 10% fetal bovine serum (FBS)-containing 1,640 medium.

(8) A resulting K562 cell suspension was completely transferred to a flask with 4 mL of a 10% FBS-containing 1,640 medium.

(9) The flask was shaken in a crossing manner, and then placed under a microscope to observe a state of cells.

(10) The flask was placed in a 5% carbon dioxide incubator to cultivate the cells at 37° C.

(11) 24 h later, the medium was changed.

2. Preparation of a single-cell suspension

3. Capture of single cells:

(1) A cultivated cell suspension with a concentration of about 1×10⁵ cells/mL was transferred to a 15 mL centrifuge tube.

(2) The cell suspension was centrifuged at 800 rpm for 3 min, and a resulting supernatant

was discarded.

(3) 5 mL of pre-cooled PBS at 4° C. was added, a resulting mixture was centrifuged at 800 rpm for 3 min, and a resulting supernatant was discarded.

(4) The above step was repeated to allow washing once again, and a resulting supernatant was discarded.

(5) The cells were resuspended with 100 μL of a pre-cooled 1,640 medium, and a resulting suspension was placed on ice.

(6) A 6-well plate or a 60 mm petri dish was prepared, and 1 mL of pre-cooled 10% FBS-containing PBS and 10 μL of the cell suspension were added.

(7) A resulting cell suspension was observed under an inverted microscope, and if a cell concentration was too high, the cell suspension was diluted appropriately until there was 1 to 2 cells in a field of view under 10× objective lens.

(8) Single cells were captured by a 10 μL long pipette tip with a filter cartridge under an inverted microscope.

(9) 1 μL of a single cell-containing solution was finally captured and transferred to a bottom of a 96-well plate or an 8-line tube for a subsequent CNV library construction experiment.

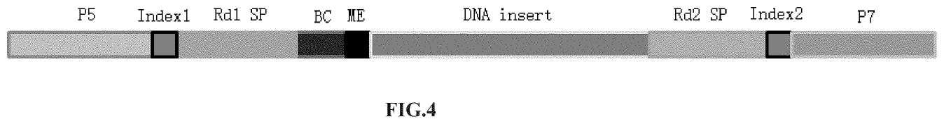

The above results were shown in FIG. 3. A 2.5 μL pipette and a 10 μL pipette tip with a filter cartridge were used in combination for screening and capture of single cells. A single cell is visible in the black circle in the field of view in this figure; and the whole intact cell is completely sucked in through a 1 μL system, and any other cells or impurities are controlled at an appropriate concentration. Therefore, basically only a single cell exists in the 1 μL system. In addition, the implementation of microscopic examination for single-cell capture with the same procedure helps us to validate the cell quality and cell number.

The above for cell preparation before library construction. In practical applications, a cell or bulk of cells from solid tissue, blood, an analytically enriched clinical sample (such as CTC enrichment or flow cytometry enrichment), directly picked sample (such as a cell obtained by a laser or a cell picked by a Tip), or collected by an organic physical, chemical, or biological method is applied as a research subject.

III. Construction of a Single-Cell Library

1. Lysis of a Single Cell:

(1) 1 μL of Zymo Genomic Lysis Buffer was added to the 1 μL single-cell-containing system.

(2) A reaction was conducted at room temperature for 10 min (at 7.5 min, a bottom of a tube was flicked 3 times with fingers for thorough mixing, and then the tube was centrifuged instantaneously),

(3) 1 μL of Thermo sterile enzyme-free water was added, and lysis was further conducted for 10 min (at 7.5 min, the bottom of the tube was flicked 3 times with fingers for thorough mixing, and then the tube was centrifuged instantaneously).

2. Purification of Single-Cell gDNA:

(1) AMPure magnetic beads (the magnetic beads needed to be equilibrated at room temperature for 30 min in advance) were added in a volume (6 μL) 2 times of a volume of the above system to the system, and a resulting mixture was incubated for 15 min.

(2) The mixture was placed on a magnetic separator to allow a reaction for 1 min to 2 min until magnetic beads with DNA adsorbed aggregated and were adsorbed by a magnet.

(3) A resulting supernatant was discarded, the magnetic beads were washed with 200 μL of 80% ethanol (this step was conducted on a magnetic separator), and a resulting supernatant was removed.

(4) The above step was repeated to wash DNA.

(5) A 200 μL pipette tip with a filter cartridge was used to remove ethanol, and then a 10 μL pipette tip with a filter cartridge was used to completely remove the residual ethanol.

(6) The magnetic separator was placed in a biosafety cabinet and air-dried for 10 min to 15 min until the magnetic beads were dry, which should prevent the magnetic beads from cracking.

3. Fragmentation and Adapter Addition for gDNA:

(1) 3 μL of sterile enzyme-free water pre-warmed to 60° C. was added to the magnetic beads, and a resulting mixture was incubated for 1 min to 2 min to make DNA dissolved.

(2) The mixture was centrifuged instantaneously, and then 1 μL of 5×LM Buffer was added.

(3) The assembled Tn5 transposome was added according to a number of single cells required for library construction, and a reaction was conducted at 55° C. for 20 min to allow nucleic acid fragmentation and addition of amplification adapter sequences (namely, the above-mentioned sequencing adapters for library construction, AC and BC).

(4) 1 μL of NT Buffer or 0.2% SDS was added to allow a reaction at 55° C. for 8 min to terminate the fragmentation reaction of the Tn5 transposase system.

4. Pooling and DNA Purification:

(1) The 8-tube strip or the 96-well plate was placed in a magnetic separator for 1 min to 2 min, and a resulting supernatant was completely transferred to a new 1.5 mL centrifuge tube.

(2) Binding Buffer (Zymo DNA concentration & purification Kit) was added in a volume 5 times a volume of the supernatant, and a resulting mixture was vortexed for 2 s to 5 s to obtain a mixed solution.

(3) 1 μL of Carrier DNA (arh35F, Sangon Biotech (Shanghai) Co., Ltd.) was added in advance to a purification column, and the purification column was incubated for 1 min.

(4) The mixed solution obtained in step (2) was transferred to the purification column and centrifuged at 12,000 rpm for 1 min. If a pooling volume was too large, a part of the mixed solution was first transferred and centrifuged, and then the remaining part was transferred and allowed to pass through the purification column until DNA in the mixed solution obtained in step (2) was completely adsorbed by the purification column. A resulting filtrate was discarded.

(5) 200 μL of Wash Buffer was added to the purification column and centrifuged at 12,000 rpm for 1 min.

(6) Step 5 was repeated.

(7) 6 μL of sterile enzyme-free water at 60° C. was added to the purification column, the centrifuge tube was replaced with a new centrifuge tube, the purification column was incubated for 1 min and then centrifuged at 12,000 rpm for 1 min.

(8) The above step was repeated until a final solution obtained in a new centrifuge tube was purified DNA.

4. PCR Amplification

A PCR system was prepared according to the table below.

TABLE 4
PCR system

| Reaction system | Volume | |
|---|---|---|
| Purified DNA | 10 μL (complete transfer, a total volume | |
| | after purification is about 10 μL) | |
| P7 primer | 2 | μL |
| P5 primer | 2 | μL |
| Gold PCR Master MIX | 86 | μL |

100 μL in total

A PCR program was set according to the table below.

TABLE 5
PCR program settings

| Temperature | | Time | | Step | Note |
|---|---|---|---|---|---|
| 105° | C. | | | | Heated lid temperature |
| 72° | C. | 3 | min | 1 | |
| 98° | C. | 30 | s | 2 | |
| 98° | C. | 15 | s | 3 | (Steps 3, 4, and 5) 23 to 27 cycles |
| 60° | C. | 30 | s | 4 | |
| 72° | C. | 3 | min | 5 | |
| 72° | C. | 5 | min | 6 | |

| | | | |
|---|---|---|---|
| 4° | C. | hold | 7 |

Notes: A number of cycles is determined according to a number of single cells pooled for library construction. Generally, 27 to 28 cycles are adopted for a single cell, and 22 to 23 cycles are adopted for pooling of 48 cells. The primers P7 and P5 are commercially-available kits, which may be purchased from Vazyme or Illumina.

5. Purification of a PCR Product

(1) Because a PCR product included impurities, it was necessary to purify the PCR product with a Zymo DNA concentration & purification Kit before E-Gel analysis,

(2) The PCR product (100 μL) was completely transferred to a new 1.5 mL centrifuge tube, 500 μL of Binding Buffer was added, and a resulting mixture was shaken for 5 s to allow thorough mixing.

(3) The mixture was completely transferred to a purification column and centrifuged at room temperature and 12,000 rpm or more for 1 min, and a resulting filtrate was discarded.

(4) 200 μL of Wash Buffer was added to the purification column, the purification column was centrifuged at room temperature and 12,000 rpm or more for 1 min, and a resulting filtrate was discarded.

(5) Step (4) was repeated.

(6) The purification column was transferred to a new 1.5 mL centrifuge tube, 10 μL of sterile enzyme-free water pre-warmed to 60° C. was added to a center of the purification column, and the purification column was centrifuged at room temperature and 12,000 rpm or more for 1 min.

(7) 10 μL of sterile enzyme-free water pre-warmed to 60° C. was added to the center of the purification column, and the purification column was centrifuged at room temperature and 12,000 rpm or more for 1 min.

(8) There was about 20 μL of a purified product in the 1.5 mL centrifuge tube, and the purified product could be immediately used for E-Gel analysis or stored at −20° C.

A purified sequencing library obtained according to the above steps has the following structure as shown in FIG. 4:

(SEQ ID NO: 54)
5′-AATGATACGGCGACCACCGAGATCTACAC
(SEQ ID NO: 52)
(index1)TCGTCGGCAGCGTCAGATGTGTATAAGAGACAG
(NNN + barcode consisting of M bases)
(SEQ ID NO: 51)
AGATGTGTATAAGAGACAG
(SEQ ID NO: 55)
TARGET-CTGTCTCTTATACACATCTCCGAGCCCACGAGAC
(index2)ATCTCGTATGCCGTCTTCTGCTTG
(SEQ ID NO: 56)-3′.

The structure is sequentially as follows from left to right (5′ to 3′): A standardized P5 adapter is first arranged to anchor a bridge PCR sequencing cell (Flow Cell) of the Illumina NGS platform, and the adapter has a specific sequence of 5′-AATGATACGGCGACCACCGAGATCTACAC-3′ (SEQ ID NO: 54). Then an index sequence index1 to recognize a sample is arranged. Rd1 SP is a sequencing primer-binding sequence for one terminus of double-terminal sequencing, and has a specific sequence of 5′-TCGTCGGCAGCGTCAGATGTGTATAAGAGACAG-3′ (SEQ ID NO: 52). BC is a barcode sequence to recognize a single cell. In the present application, three random nucleotides NNN are added in front of the recognition sequence to prevent an initial signal from being unstable during sequencing to cause a decrease in a recognition rate of the barcode. Then an anchor sequence (an ME sequence) is arranged to locate the barcode sequence and simulate an ME sequence AGATGTGTATAAGAGACAG (SEQ ID NO: 51), and the anchor sequence usually binds to and be assembled with the Tn5 transposase. The gray DNA insert in FIG. 4 represents a DNA fragment to be sequenced. Rd2 SP is a sequencing primer-binding sequence for the other terminus of double-terminal sequencing. An index sequence 2 (index2) is a tag sequence at a P7 terminus.

This sequence is designed to reduce a cost, be efficient, and match the existing sequencing platform, and thus double-terminal sequencing and double-terminal indexes are adopted. Because the sequencing read primer and indexes included at the P5 and P7 termini match the existing sequencing platform, an amount of sequencing data is determined according to needs. There is no need to pack all lanes or flow cell, which reduces a cost of sequencing to some extent.

6. E-GEL Analysis

(1) In this experiment, 2% precast gel (E-Gel) of Invitrogen was adopted, which was directly unpacked and arranged on an exclusive instrument during use, and a sample belonging to a swimming lane is marked on a gel plate,

(2) loading of samples: If 50 bp DNA Marker (Thermo Fisher, Cat. No. 10488099) was adopted, it was necessary to add 16 μL of sterile enzyme-free water and 4 μL of Maker to each of two Maker wells (because the Maker wells were at two sides, a small amount of a liquid would be leaked out sometimes, in which case the well should be filled with sterile enzyme-free water to 20 μL). If another Marker was adopted, 20 μL of a solution could be directly added. According to different operating habits and operating skills, when a sample was added, two sample wells needed to be spaced by a well to prevent two samples from contaminating each other during gel extraction and electrophoresis. 20 μL of the purified product was added to the gel plate, and the spacer well needed to be filled with sterile enzyme-free water to 20 μL. If a sample was of less than 20 μL, it was necessary to add sterile enzyme-free water to 20 μL.

(3) Electrophoresis: In order to verify the construction of a library and recover a 300 bp to 500 bp DNA fragment, 0.8% to 2% precast gel generally needed to run for 18 min until a 50 bp DNA fragment at a Marker band ran to a black adhesive tape close to an E-Gel packing plate.

(4) Preliminary observation results: A gel fluorescence imaging system was used to observe bands for construction of a sequencing library and acquire an image for recording.

(5) Gel extraction: A 300 bp to 500 bp DNA fragment was cut off.

(6) A gel in a recovery zone was cut off and added to a 1.5 mL centrifuge tube, weighed, and then used in a subsequent gel purification step or stored at 4° C.

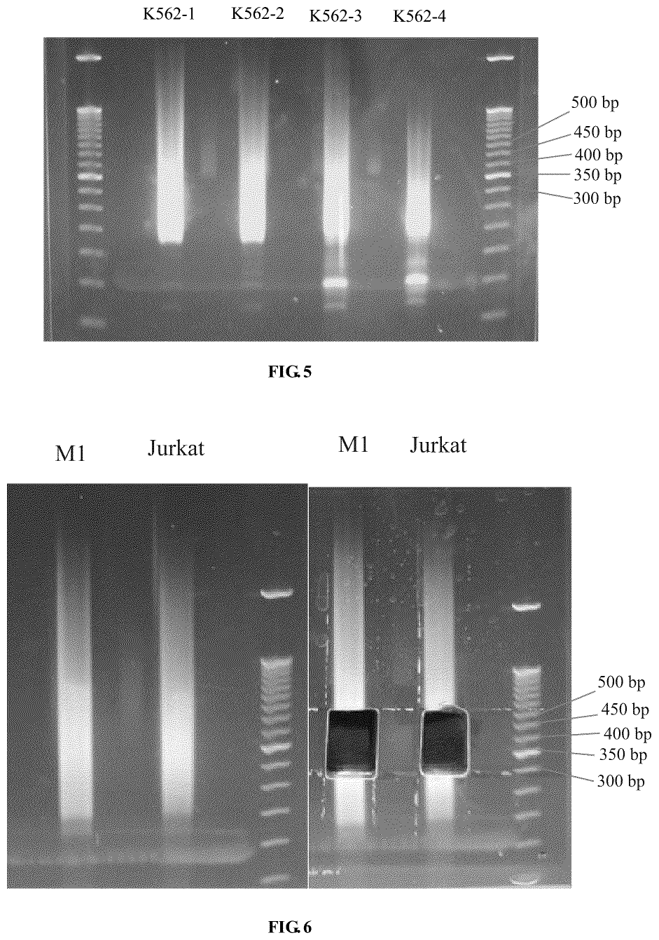

The above experimental results were shown in FIG. 5 and FIG. 6. Bands in the figures are bright, indicating successful preparation of a library.

7. Gel Recovery and Purification of DNA

(1) A Zymo Gel Purification Kit was used to recover and purify a DNA fragment in a gel.

(2) The recovered gel was added to AD buffer according to a ratio of 1:3 (namely, 1 mg: 3 mL) (a 300 bp to 500 bp DNA fragment was generally 0.9 mg, 270 μL of AD buffer was added, and a gel of each lane was placed in a separate 1.5 mL centrifuge tube).

(3) A reaction was conducted in a 55° C. metal bath for 15 min until the gel was completely dissolved.

(4) A resulting solution was completely transferred to a chromatographic column and centrifuged at room temperature and 10,000 rpm or more for 1 min, and then a resulting filtrate was discarded.

(5) 200 μL of Wash Buffer was added to the chromatographic column, the chromatographic column was centrifuged at room temperature and 10,000 rpm or more for 1 min, and a resulting filtrate was discarded.

(6) Step 4 was repeated.

(7) The purification column was transferred to a new 1.5 mL centrifuge tube, 8 μL of sterile enzyme-free water pre-warmed to 60° C. was added to a center of the purification column, and the purification column was centrifuged at room temperature and 10,000 rpm or more for 1 min.

(8) 10 μL of sterile enzyme-free water pre-warmed to 60° C. was added to the center of the purification column, and the purification column was centrifuged at room temperature and 10,000 rpm or more for 1 min.

(9) There was about 16 μL of a purified product in the 1.5 mL centrifuge tube, and the purified product could be detected by an Agilent 2100 nucleic acid analyzer and Qit or stored at −20° C. before being used in the next sequencing step.

8. Concentration Detection by a Qubit 3.0 Fluorometer Nucleic Acid Analyzer

(1) Standardization instrument: Two tubes were taken; 199 μL of Working Buffer was added to each tube, and then 1 μL of a fluorescent dye was added to the tube; the tubes each were centrifuged instantaneously and then vortexed for thorough mixing; 10 μL of a liquid in each tube was discarded through a pipette tip, and then 10 μL of a standard reagent was added; the tubes each were centrifuged instantaneously, then vortexed for thorough mixing, statically incubated at room temperature for 2 min, and then placed in an instrument; and a screen button of a manipulator was clicked to allow an automatic standardization operation.

(2) Measurement of a concentration: A corresponding number of matching centrifuge tubes were taken; 199 μL of Working Buffer was added to each tube, and then 1 μL of a fluorescent dye was added to the tube; and the tubes were labeled, vortexed for thorough mixing, and centrifuged instantaneously.

(3) 1 μL of a liquid in each centrifuge tube was discarded, then 1 μL of a sample was added to the centrifuge tube, and the centrifuge tube was vortexed for thorough mixing, centrifuged instantaneously, statically incubated at room temperature for 2 min, and placed in an instrument.

(4) ds DNA was selected, and according to instructions of a panel, a dilution factor was adjusted, and a final concentration of DNA in a library was detected.

The above experimental results were shown in the table below:

TABLE 6
Concentration analysis results of a Qubit nucleic acid analyzer
for construction of MT-scCNV-seq libraries of K562 cell line

| Batch name | Qubit concentration (ng/μL) | Note |
|---|---|---|
| 1.0313p8 | 6.4 | |
| 2.0318p8 | 17.4 | |
| 3.0324p24 | 13.1 | |
| 4.0325p24 | 9.86 | |
| 5.0402p24 | 3.88 | |
| 6.0411p24 | 2.74 | |
| 7.0418p8 | 2.08 | |

Summation: Theoretical concentration after pooling: 4.844 ng/μL; and sequencing concentration: 5.94 ng/μL

TABLE 7
Concentration analysis results of a Qubit nucleic
acid analyzer for construction of a pooled library
of a Jurkat cell line and a normal human PBMC

| Batch name | Qubit concentration (ng/μL) | Note |
|---|---|---|
| M1-1(Normal) | 3.84 | |
| M1-3 | 5.00 | |
| M1-4 | 4.24 | |
| M1-5 | 3.72 | |
| JK1 | 3.86 | |
| JK2 | 3.32 | |
| MIX1(M1 + JK) | 6.12 | |
| MIX2 | 6.50 | |
| MIX3 | 6.96 | |

Summation: Theoretical concentration after pooling: 4.91 ng/μL; and sequencing concentration: 6.16 ng/μL

TABLE 8
Concentration analysis results of a Qubit nucleic acid analyzer
for construction of a GM12878 cell line library

| | | |
|---|---|---|
| Batch name | Qubit concentration | Note |

| | |
|---|---|
| GM12878 | 6.96 ng/μL; and sequencing |
| | concentration: 4.18 ng/μL |

Since a quality of library construction needs to be determined before sequencing, a Qubit nucleic acid analyzer developed by Invitrogen needs to be used for concentration detection. According to the results in the table above, libraries constructed from the above cells all meet the requirement of a sequencing concentration of 2 ng/μL.

9. Analysis by an Agilent 2100 Nucleic Acid Analyzer

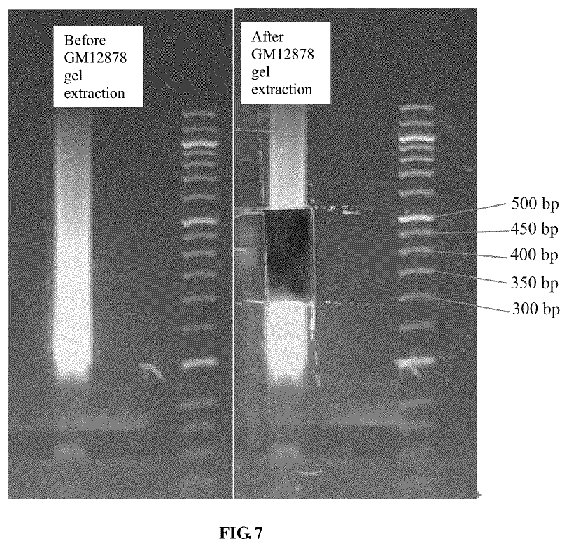

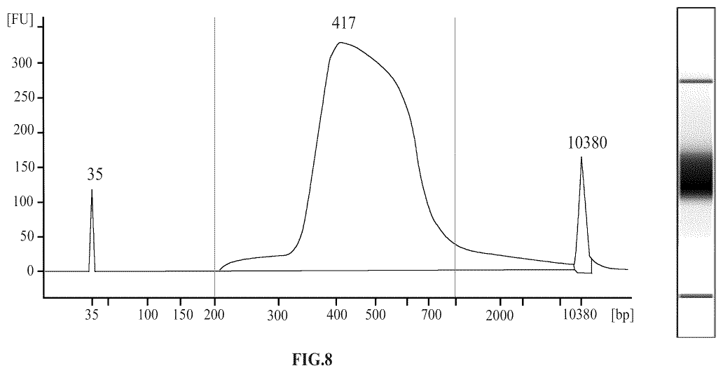

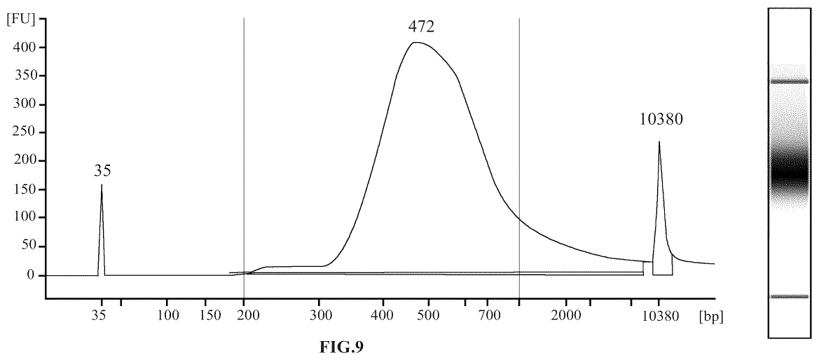

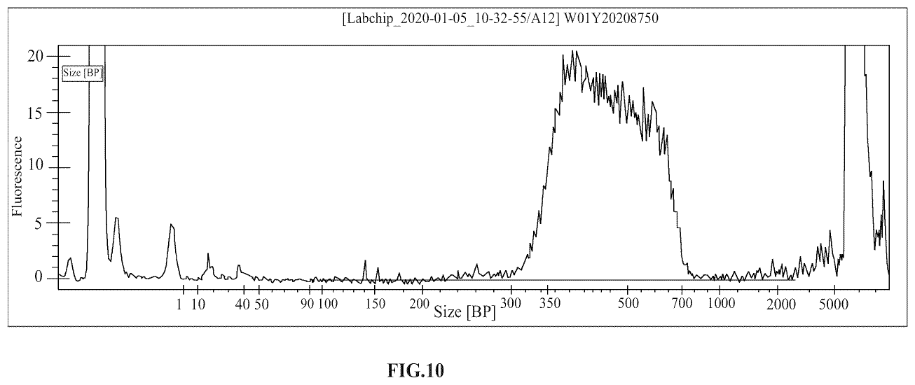

The above experimental results were shown in FIG. 7 to FIG. 9. Fragments in the single-cell CNV libraries constructed by the method of the present application for the K562 cell line (a total of 120 single cells), the normal control group, the Jurkat cell line (a total of 96 single cells), and the GM12878 cell line (a total of 48 single cells) were detected by Agilent 2100, and the kurtosis was 300 bp to 800 bp (as shown in FIG. 10), which met the standards of sequencing on a computer.

Quality analysis results of sequencing data were shown in Tables 9 to 11.

TABLE 9
Data quality of MT-scCNV-seq library for single cells
of the K562 cell line (with one set as an example)

| | scCNV-seq library for |
|---|---|
| Sample | the K562 cell line |
| Raw Read Number | 201270154 |
| Raw Base Number | 30190523100 |
| Clean Read Number | 198494662 |
| Clean Read Rate (%) | 98.6200 |
| Clean Base Number | 29774199300 |
| Low-quality Read Number | 1221474 |
| Low-quality Read Rate (%) | 0.6100 |
| Ns Read Number | 0 |
| Ns Read Rate (%) | 0.0000 |
| Adapter Polluted Read Number | 1554018 |
| Adapter Polluted Read Rate (%) | 0.7700 |
| PolyG Read Number | 0.0000 |
| PolyG Read Rate (%) | 0.0000 |
| Raw Q30 Base Rate (%) | 91.1100 |
| Clean Q30 Base Rate (%) | 91.5100 |

It is seen from the table above that the data quality of the library for the K562 cell line constructed by the method generally meets an expected standard. In order to avoid data waste and determine whether double-terminal indexes of commercial standards match the method, 7 indexes are added to the same batch of cells for library construction. It is seen from Table 9 that the Clean Read Rate accounts for 98.62% of the total data volume, and Q30 Base Rates of both Raw Data and Clean Data reach 91% or more. Therefore, a quality of the library constructed by the method is in line with the requirements of later bioinformatics analysis, which leads to less data redundancy and reduces a cost.

TABLE 10
Data quality of MT-scCNV-seq libraries for single cells of Jurkat
cell line and normal human PBMC (with one set as an example)

| Sample | scCNV-seq library for a Jurkat cell line |
|---|---|
| Raw Read Number | 143496126 |
| Raw Base Number | 21524418900 |
| Clean Read Number | 140742654 |
| Clean Read Rate (%) | 98.0800 |
| Clean Base Number | 21111398100 |
| Low-quality Read Number | 1301154 |
| Low-quality Read Rate (%) | 0.9100 |
| Ns Read Number | 14766 |
| Ns Read Rate (%) | 0.0100 |
| Adapter Polluted Read Number | 1437552 |
| Adapter Polluted Read Rate (%) | 1.0000 |
| Raw Q30 Base Rate (%) | 91.1100 |
| Clean Q30 Base Rate (%) | 91.5100 |

In order to verify whether different cell lines is distinguished by barcodes and whether there is inter-cell contamination in the pooling library construction, 48 jurkat cells and 48 normal human PBMCs were used in this experiment for pooled library construction. It can be seen from Table 10 that a total data amount is about 120 G, the clean read rate is basically about 98%, and the Q30 percentage is 91%, indicating that the data is reliable, there is basically no cross-contamination. This data is qualified for the downstream bioinformatics analysis.

TABLE 11
Data quality of MT-scCNV-seq libraries for
single cells of the GM12878 cell line

| | scCNV-seq library for |
|---|---|
| Sample | the GM12878 cell line |
| Raw Read Number | 414676402 |
| Raw Base Number | 62201460300 |
| Clean Read Number | 412539208 |
| Clean Read Rate (%) | 99.4800 |
| Clean Base Number | 61880881200 |
| Low-quality Read Number | 1223078 |
| Low-quality Read Rate (%) | 0.3000 |
| Ns Read Number | 306060 |
| Ns Read Rate (%) | 0.0700 |
| Adapter Polluted Read Number | 598056 |
| Adapter Polluted Read Rate (%) | 0.1400 |
| Raw Q30 Base Rate (%) | 90.6100 |
| Clean Q30 Base Rate (%) | 90.7400 |

In order to verify whether a barcode is detected in a batch of sequencing data (with the same index on the raw sequencing reads) and test a docked sequencing platform, 48 GM12878 cells were used this time for construction of a single-batch scCNV-seq library, and an Illumina NovaSeq 6000 PE150 platform was used for sequencing of the single-batch data. The expected target data amount is 48 G, and the actual output data amount is 62 G. It is seen from Table 11 that the quality of this batch of data is excellent; the Clean Read Rate is as high as 99.48%; there is basically no adapter contamination; the Q30 is 90.7% or more.

Primer A in this embodiment is shown in Table 12 below. In this table, a lowercase part represents a self-designed barcode sequence.

TABLE 12
Primer A

| SEQ ID NO: | Sequence (5′ to 3′) |
|---|---|
| 1 | TCGTCGGCAGCGTCAGATGTGTATAAGAGACAGNNNtcgccttaAGATGTGTATAAGAGACAG |
| 2 | TCGTCGGCAGCGTCAGATGTGTATAAGAGACAGNNNctagtacgAGATGTGTATAAGAGACAG |
| 3 | TCGTCGGCAGCGTCAGATGTGTATAAGAGACAGNNNttctgcctAGATGTGTATAAGAGACAG |
| 4 | TCGTCGGCAGCGTCAGATGTGTATAAGAGACAGNNNgctcaggaAGATGTGTATAAGAGACAG |
| 5 | TCGTCGGCAGCGTCAGATGTGTATAAGAGACAGNNNaggagtccAGATGTGTATAAGAGACAG |
| 6 | TCGTCGGCAGCGTCAGATGTGTATAAGAGACAGNNNcatgcctaAGATGTGTATAAGAGACAG |
| 7 | TCGTCGGCAGCGTCAGATGTGTATAAGAGACAGNNNgtagagagAGATGTGTATAAGAGACAG |
| 8 | TCGTCGGCAGCGTCAGATGTGTATAAGAGACAGNNNcagcctcgAGATGTGTATAAGAGACAG |
| 9 | TCGTCGGCAGCGTCAGATGTGTATAAGAGACAGNNNtgcctcttAGATGTGTATAAGAGACAG |
| 10 | TCGTCGGCAGCGTCAGATGTGTATAAGAGACAGNNNtcctctacAGATGTGTATAAGAGACAG |
| 11 | TCGTCGGCAGCGTCAGATGTGTATAAGAGACAGNNNtcatgagcAGATGTGTATAAGAGACAG |
| 12 | TCGTCGGCAGCGTCAGATGTGTATAAGAGACAGNNNcctgagatAGATGTGTATAAGAGACAG |
| 13 | TCGTCGGCAGCGTCAGATGTGTATAAGAGACAGNNNtagcgagtAGATGTGTATAAGAGACAG |
| 14 | TCGTCGGCAGCGTCAGATGTGTATAAGAGACAGNNNgtagctccAGATGTGTATAAGAGACAG |
| 15 | TCGTCGGCAGCGTCAGATGTGTATAAGAGACAGNNNtactacgcAGATGTGTATAAGAGACAG |
| 16 | TCGTCGGCAGCGTCAGATGTGTATAAGAGACAGNNNaggctccgAGATGTGTATAAGAGACAG |
| 17 | TCGTCGGCAGCGTCAGATGTGTATAAGAGACAGNNNgcagcgtaAGATGTGTATAAGAGACAG |
| 18 | TCGTCGGCAGCGTCAGATGTGTATAAGAGACAGNNNctgcgcatAGATGTGTATAAGAGACAG |
| 19 | TCGTCGGCAGCGTCAGATGTGTATAAGAGACAGNNNgagcgctaAGATGTGTATAAGAGACAG |
| 20 | TCGTCGGCAGCGTCAGATGTGTATAAGAGACAGNNNcgctcagtAGATGTGTATAAGAGACAG |
| 21 | TCGTCGGCAGCGTCAGATGTGTATAAGAGACAGNNNgtcttaggAGATGTGTATAAGAGACAG |
| 22 | TCGTCGGCAGCGTCAGATGTGTATAAGAGACAGNNNactgatcgAGATGTGTATAAGAGACAG |
| 23 | TCGTCGGCAGCGTCAGATGTGTATAAGAGACAGNNNtagctgcaAGATGTGTATAAGAGACAG |
| 24 | TCGTCGGCAGCGTCAGATGTGTATAAGAGACAGNNNgacgtcgaAGATGTGTATAAGAGACAG |
| 25 | TCGTCGGCAGCGTCAGATGTGTATAAGAGACAGNNNctctctatAGATGTGTATAAGAGACAG |
| 26 | TCGTCGGCAGCGTCAGATGTGTATAAGAGACAGNNNtatcctctAGATGTGTATAAGAGACAG |
| 27 | TCGTCGGCAGCGTCAGATGTGTATAAGAGACAGNNNgtaaggagAGATGTGTATAAGAGACAG |
| 28 | TCGTCGGCAGCGTCAGATGTGTATAAGAGACAGNNNactgcataAGATGTGTATAAGAGACAG |
| 29 | TCGTCGGCAGCGTCAGATGTGTATAAGAGACAGNNNaaggagtaAGATGTGTATAAGAGACAG |
| 30 | TCGTCGGCAGCGTCAGATGTGTATAAGAGACAGNNNctaagcctAGATGTGTATAAGAGACAG |
| 31 | TCGTCGGCAGCGTCAGATGTGTATAAGAGACAGNNNcgtctaatAGATGTGTATAAGAGACAG |
| 32 | TCGTCGGCAGCGTCAGATGTGTATAAGAGACAGNNNtctctccgAGATGTGTATAAGAGACAG |
| 33 | TCGTCGGCAGCGTCAGATGTGTATAAGAGACAGNNNtcgactagAGATGTGTATAAGAGACAG |
| 34 | TCGTCGGCAGCGTCAGATGTGTATAAGAGACAGNNNttctagctAGATGTGTATAAGAGACAG |
| 35 | TCGTCGGCAGCGTCAGATGTGTATAAGAGACAGNNNcctagagtAGATGTGTATAAGAGACAG |
| 36 | TCGTCGGCAGCGTCAGATGTGTATAAGAGACAGNNNgcgtaagaAGATGTGTATAAGAGACAG |
| 37 | TCGTCGGCAGCGTCAGATGTGTATAAGAGACAGNNNctattaagAGATGTGTATAAGAGACAG |
| 38 | TCGTCGGCAGCGTCAGATGTGTATAAGAGACAGNNNaaggctatAGATGTGTATAAGAGACAG |
| 39 | TCGTCGGCAGCGTCAGATGTGTATAAGAGACAGNNNgagccttaAGATGTGTATAAGAGACAG |
| 40 | TCGTCGGCAGCGTCAGATGTGTATAAGAGACAGNNNttatgcgaAGATGTGTATAAGAGACAG |
| 41 | TCGTCGGCAGCGTCAGATGTGTATAAGAGACAGNNNtatagcctAGATGTGTATAAGAGACAG |
| 42 | TCGTCGGCAGCGTCAGATGTGTATAAGAGACAGNNNatagaggcAGATGTGTATAAGAGACAG |
| 43 | TCGTCGGCAGCGTCAGATGTGTATAAGAGACAGNNNcctatcctAGATGTGTATAAGAGACAG |
| 44 | TCGTCGGCAGCGTCAGATGTGTATAAGAGACAGNNNggctctgaAGATGTGTATAAGAGACAG |
| 45 | TCGTCGGCAGCGTCAGATGTGTATAAGAGACAGNNNaggcgaagAGATGTGTATAAGAGACAG |
| 46 | TCGTCGGCAGCGTCAGATGTGTATAAGAGACAGNNNtaatcttaAGATGTGTATAAGAGACAG |
| 47 | TCGTCGGCAGCGTCAGATGTGTATAAGAGACAGNNNcaggacgtAGATGTGTATAAGAGACAG |
| 48 | TCGTCGGCAGCGTCAGATGTGTATAAGAGACAGNNNgtactgacAGATGTGTATAAGAGACAG |

Primer B in this embodiment:

(SEQ ID NO: 49)
5′-GTCTCGTGGGCTCGAGATGTGTATAAGAGACAG-3′.

Primer C in this embodiment:

(SEQ ID NO: 50)
5′ Phos-CTGTCTCTTATACACATCT-3′,
wherein “phos” represents phosphorylation.

The above embodiments are merely intended to illustrate some implementations of the present application in detail, and should not be considered as a limitation to the scope of the present application. It should be noted that those of ordinary skill in the art further may make several alternations and improvements without departing from the concept of the present application, and these alternations and improvements all fall within the protection scope of the present application. Therefore, the protection scope of the present application should be subject to the protection scope defined by the claims.

1. A method for construction of a medium-throughput single-cell copy number sequencing (MT-scCNV-seq) library, comprising:
providing sorted single cells;
independently lysing each single cell to fully expose a genomic DNA (gDNA) of the single cells;
tagmenting the gDNA and conducting sample-specific DNA labeling to obtain the whole set of fragmented gDNAs labeled with a cell-specific barcode in a given cell, while each cell has a different barcode; and
pooling the labeled fragmented gDNAs of a plurality of single cells to collectively construct a MT-scCNV-seq library for subsequent sequencing,
wherein Tn5 transposome is used to tagment the gDNA in the single cell and label each gDNA fragments with a barcode; and
further, after next-generation sequencing (NGS) is completed with the constructed sequencing library, data output is analyzed by a relevant program and method to determine the DNA copy number profile over the whole genome of each cell.

2. The method according to claim 1, wherein the Tn5 transposome comprises Tn5 transposase and two double strands of oligonucleotides, while one double strand of oligonucleotides Tn5P5 adapter is annealed from primer A and primer C, and the other double strand of oligonucleotides Tn5P7 adapter is annealed from primer B and primer C;
the primer A comprises a cell barcode labeling sequence consisting of 3 to 23 single nucleotides, P5 PCR handle sequence, and reverse mosaic end (ME) sequence;
the primer B comprises P7 PCR handle sequence and the reverse ME sequence; and
the primer C is an oligonucleotide with a phosphorylated 5′ terminus, and is partially complementary to each of the primer A and the primer B.

3. The method according to claim 1, wherein the Tn5 transposome comprises Tn5 transposase and two double strands of oligonucleotides, while one double strand of oligonucleotides Tn5P5 adapter is annealed from primer A and primer C, and the other double strand of oligonucleotides Tn5P7 adapter is annealed from primer B and primer C;
the primer A comprises a cell barcode labeling sequence consisting of 3 to 23 single nucleotides, P7 PCR handle sequence, and the reverse ME sequence;
the primer B comprises P5 PCR handle sequence and the reverse ME sequence; and
the primer C is an oligonucleotide with a phosphorylated 5′ terminus, and is partially complementary to each of the primer A and the primer B.

4. The method according to claim 2, wherein the primer A has a nucleotide sequence shown in SEQ ID NO: 1-48.

5. The method according to claim 2, wherein the primer B has a nucleotide sequence shown in SEQ ID NO: 49.

6. The method according to claim 2, wherein the primer C has a nucleotide sequence shown in SEQ ID NO: 50.

7. The method according to claim 1, further comprising the following steps:
(1) adding multiple single cell each to a different independent single tube;
(2) lysing each single cell in its tube with a lysis buffer or protease;
(3) inactivating the protease and optionally purifying the lysate or diluting the lysate to eliminate any factor from inhibition on the subsequent reaction;
(4) using the Tn5 transposome to tagment the gDNA obtained after lysing the single cell, and adding a cell-specific barcode recognition sequence consisting of 3 to 23 single nucleotides to the gDNA;
(5) pooling fragmented gDNA samples of a plurality of single cells in a single tube, and purifying the fragmented gDNA samples, and then concentrating the fragmented gDNA samples;
(6) subjecting the concentrated gDNA samples in the single tube as a batch of samples to polymerase chain reaction (PCR) amplification to construct a multi-sample library of this batch of single cells in parallel in the single tube, wherein PCR amplification primers that comprise a specific batch index sequence and are compatible with an NGS system are adopted for each batch of gDNA samples; and
(7) purifying the multi-sample library, and recovering an aimed range of DNA sizes for the multi-sample library, with the size range varies from 300 bp-1000 bp or any range in between.

8. The method according to claim 7, wherein in step (6), an anchor sequence and a cell barcode sequence are added to a 5′ terminus of each insert DNA fragment, and subsequently, when the DNA fragment is amplified, an amplification adapter sequence compatible with a NGS sequencing system is added to each of upstream and downstream primers for the amplification; and
an amplified DNA fragment from 5′ terminus to 3′ terminus consequently comprises the P5 adapter sequence, the first index sequence, the first sequencing primer binding site, the cell barcode sequence, the anchor sequence, the insert DNA fragment, the second sequencing primer binding site, the second index sequence, and the P7 adapter sequence, and all amplified DNA fragments constitute an library compatible with the NGS sequencing system.

9. The method according to claim 8, wherein the NGS sequencing system is an Illumina sequencing system or another sequencing system.

10. The method according to claim 8, wherein the cell barcode sequence is an oligonucleotide with 3 to 23 nucleotides comprising 2 to 5 random nucleotides and 1 to 18 nucleotides constituting a barcode.

11. The method according to claim 8, wherein the anchor sequence is 5′-AGATGTGTATAAGAGACAG-3′ (SEQ ID NO: 51).

12. The method according to claim 8, wherein in the NGS library, 5′-AATGATACGGCGACCACCGAGATCTACAC(SEQ ID NO: 54) (index1)TCGTCGGCAGCGTCAGATGTGTATAAGAGACAG (SEQ ID NO: 52) (NNN+barcode consisting of M bases)AGATGTGTATAAGAGACAG (SEQ ID NO: 51)-TARGET-CTGTCTCTTATACACATCTCCGAGCCCACGAGAC(SEQ ID NO: 55) (index2)ATCTCGTATGCCGTCTTCTGCTTG (SEQ ID NO: 56)-3′, wherein “TARGET” represents the DNA fragment to be tested, “N” represents any one selected from the group consisting of bases A, T, C, and G, and “M” is 1 to 18.

13. The method according to claim 1, wherein the single cell is replaced with a micro-bulk of cells, and the micro-bulk cells refer to 2 to 50, 50 to 100, 100 to 200, 200 to 500, or 500 to 1000 cells.

14. The method according to claim 1, wherein the single cell is replaced with gDNA, and an amount of the gDNA is 1 pg to 1 μg.

15. The method according to claim 7, wherein in step (2), the sorted single cell or micro-bulk cells in the tube is/are lysed with a detergent-containing lysis buffer or a Zymo genomic lysis buffer or a Qiagen protease.

16. The method according to claim 1, wherein the relevant program and method for analyzing the data output to determine the copy number comprises analysis software, an algorithm, a database, a website, and a visualization scheme.

17. A method of basic research, clinical screening, diagnosis, treatment, and drug research and development for a tumor, comprising:
constructing a copy number sequencing library of single cells or micro-bulk cells or corresponding gDNAs of a target subject; and
sequencing the copy number sequencing library,
wherein the copy number sequencing library is constructed by the method according to claim 1; and
the single cells or micro-bulk cells of the target subject are derived from a solid tumor tissue, a leukemia sample, circulating tumor cells (CTCs), a minimal residual disease (MRD) sample, a fine needle aspiration biopsy sample, a hydrothorax (usually caused by lung cancer) sample, a hydroperitoneum (usually caused by tumors in abdomen) sample, a urine sample, a vaginal sample, a cervical sample, or a cerebrospinal fluid, or the single cells of the target subject are single cells from a subject of another liquid biopsy or a surgical treatment.

18. A method of basic research, clinical screening, diagnosis, treatment, and drug research and development for fertility and reproduction genetics, comprising:
constructing a copy number sequencing library of single cells or micro-bulk cells or corresponding gDNAs of a target subject; and
sequencing the copy number sequencing library,
wherein the copy number sequencing library is constructed by the method according to claim 1; and
the single cells or micro-bulk cells of the target subject are derived from a non-invasive prenatal test (NIPT) subject, a prenatal diagnosis (PD) subject, a preimplantation genetic test (PGT) subject, or a genetic test of miscarriage product subject.

19. A hardware system for high-throughput (HT) gDNA copy number sequencing, comprising:
a microfluidic chip, or
a cell recognition, enrichment, and sorting system, or
an automated liquid delivering system, and
a computer software program configured to implement the hardware system,
wherein the microfluidic chip or the cell recognition, enrichment, and sorting system is configured to sort and acquire target single cells and construct a sequencing library, and the sequencing library is constructed by the method according to claim 1.
